(19) United States
(12) Patent Application Publication
(10) Pub. No.: US 2026/0248452 A1
(43) Pub. Date: Aug. 27, 2026

(54) ELECTRONIC DEVICE FOR OBTAINING BIOMETRIC INFORMATION AND METHOD OF CONTROLLING THE SAME

(71) Applicant: Samsung Electronics Co., Ltd., Suwon-si (KR)

(72) Inventors: Jaemin KANG, Suwon-si (KR); Joongwoo AHN, Suwon-si (KR)

(21) Appl. No.: 19/439,101

(22) Filed: Jan. 2, 2026

Related U.S. Application Data
(63) Continuation of application No. PCT/KR2025/022871, filed on Dec. 26, 2025.

(30) Foreign Application Priority Data
Feb. 27, 2025 (KR) 10-2025-0025642
Mar. 31, 2025 (KR) 10-2025-0041263

(51) Int. Cl. A61B 5/00

(52) U.S. Cl. CPC A61B 5/6844; A61B 5/0059; A61B 5/681

(57) ABSTRACT

An electronic device may include: a first light emitting device configured to emit light, a first light receiving device configured to receive light of a first wavelength band, a second light receiving device configured to receive light of a second wavelength band, a memory including at least one storage medium storing instructions, and at least one processor, comprising processing circuitry, wherein at least one processor, individually and/or collectively, is configured to execute the instructions and to cause the electronic device to receive first light by the first light receiving device in response to light emission by the first light emitting device, receive second light by the second light receiving device in response to light emission by the first light emitting device, and determine whether the electronic device contacts at least a portion of a body of a user based on the first light and the second light.

### CROSS-REFERENCE TO RELATED APPLICATIONS

This application is a continuation of International Application No. PCT/KR2025/022871 designating the United States, filed on Dec. 26, 2025, in the Korean Intellectual Property Receiving Office and claiming priority to Korean Patent Application Nos. 10-2025-0025642, filed on Feb. 27, 2025, and 10-2025-0041263, filed on Mar. 31, 2025, in the Korean Intellectual Property Office, the disclosures of each of which are incorporated by reference herein in their entireties.

### BACKGROUND

### Field

The disclosure relates to an electronic device that obtains biometric information and a control method thereof.

### Description of Related Art

As electronic devices become smaller and lighter, various types of electronic devices that may be worn on a user's body have emerged. Wearable electronic devices have high portability and high proximity to a user's body, so they may be utilized for various purposes. A wearable electronic device may include (e.g., be equipped with) a plurality of sensors (e.g., a proximity sensor, a temperature sensor, a biometric sensor) for measuring and obtaining biometric information from a user. For example, a wearable electronic device may obtain biometric information such as an electrocardiogram (ECG), respiration, electromyography (EMG), electrooculography (EOG), electroencephalogram (EEG), blood glucose, oxygen saturation (SpO₂), photoplethysmogram (PPG), body temperature, and other various information.

The above-described information may be provided as related art for the purpose of helping understanding of the disclosure. No assertion or determination is made as to whether any of the foregoing is applicable as background art in relation to the disclosure.

### SUMMARY

Embodiments of the disclosure provide an electronic device that obtains biometric information and a control method thereof. In an example embodiment, an electronic device and a control method thereof may determine whether the electronic device contacts at least a portion or a part of a user's body. In an example embodiment, an electronic device and a control method thereof may measure and obtain biometric information based on determining that the electronic device contacts at least a portion of the user's body.

According to an example embodiment, an electronic device may include: a first light emitting device configured to emit light, a first light receiving element device configured to receive light of a first wavelength band, a second light receiving device configured to receive light of a second wavelength band, a memory including at least one storage medium storing instructions, and at least one processor, comprising processing circuitry, wherein at least one processor, individually and/or collectively, may be configured to execute the instructions and to cause the electronic device to perform at least one operation. The at least one operation may include receiving first light by the first light receiving device in response to light emission by the first light emitting device. The at least one operation may include receiving second light by the second light receiving device in response to light emission by the first light emitting device. The at least one operation may include determining whether the electronic device contacts at least a portion of a user's body based on the first light and the second light.

According to an example embodiment, a method of controlling an electronic device may include at least one operation. The at least one operation may include receiving first light by a first light receiving device in response to light emission by a first light emitting device. The at least one operation may include receiving second light by a second light receiving device in response to light emission by a first light emitting device. The at least one operation may include determining whether an electronic device contacts at least a portion of a user's body based on first light and second light.

According to an example embodiment, a non-transitory computer readable storage medium stores at least one instruction, wherein the at least one instruction, when executed by at least one processor, comprising processing circuitry, of an electronic device, individually and/or collectively, may cause the electronic device to perform at least one operation. The at least one operation may include receiving first light by a first light receiving device in response to light emission by a first light emitting device. The at least one operation may include receiving second light by a second light receiving device in response to light emission by a first light emitting device. The at least one operation may include determining whether an electronic device contacts at least a portion of a user's body based on first light and second light.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a block diagram illustrating an example electronic device in a network environment according to an embodiment.

FIGS. 2A and 2B are a diagram and a graph illustrating an example method of obtaining biometric information using an optical sensor, according to an embodiment.

FIG. 3A is a front perspective view illustrating an example electronic device according to an embodiment.

FIG. 3B is a rear perspective view illustrating an example electronic device according to an embodiment.

FIG. 4 is a an exploded perspective view illustrating an example in which an optical sensor is disposed in an electronic device according to an embodiment.

FIG. 5 is a perspective view illustrating various regions on a circuit board divided by a barrier member disposed on the circuit board, according to an embodiment.

FIG. 6A is a diagram illustrating various elements of an optical sensor disposed in a plurality of regions divided by a barrier member, according to an embodiment.

FIG. 6B is a cross-sectional view illustrating a plurality of regions divided by a barrier member, according to an embodiment.

FIG. 6C is a diagram illustrating light received by a light receiving element based on light emitted from a light emitting element according to an embodiment.

FIG. 6D is a cross-sectional view illustrating a light-filtering member of an electronic device according to an embodiment.

FIG. 6E is a cross-sectional view illustrating two light-filtering members of an electronic device according to an embodiment.

FIG. 7 is a block diagram illustrating an example configuration for biometric information acquisition, according to an embodiment.

FIGS. 8A, 8B, 8C and 8D are graphs illustrating light received by a light receiving element, according to an embodiment.

FIGS. 9A and 9B are graphs illustrating an example method of determining a contact state using a pair of optical signals, according to an embodiment.

FIGS. 10A, 10B, 10C and 10D are graphs illustrating light obtained by a light receiving element, according to an embodiment.

FIG. 11 is a graph illustrating an example method of determining a contact state using a pair of optical signals, according to an embodiment.

FIG. 12 is a graph illustrating light received by a light receiving element, according to an embodiment.

FIG. 13 is a flowchart illustrating example operations of an electronic device according to an embodiment.

FIG. 14 is a flowchart illustrating example operations of an electronic device according to an embodiment.

FIG. 15 is a diagram illustrating feedback provided by an electronic device according to an embodiment.

FIG. 16 is a diagram illustrating example biometric information provided by an electronic device according to an embodiment.

FIG. 17 is a diagram illustrating an example ring-type electronic device according to an embodiment.

### DETAILED DESCRIPTION

Hereinafter, various example embodiments of the disclosure are described in greater detail with reference to the drawings. However, the disclosure may be implemented in other various forms and is not limited to the example embodiments set forth herein. The same or similar reference denotations may be used to refer to the same or similar elements throughout the disclosure. Further, for clarity and brevity, no description may be made of well-known functions and configurations in the drawings and relevant descriptions.

As used herein, the terms ‘light emitting element’ and ‘light receiving element’ each refer to a structural component and are intended to encompass and correspond to the ‘light emitting device’ and ‘light receiving device’ recited in the claims.

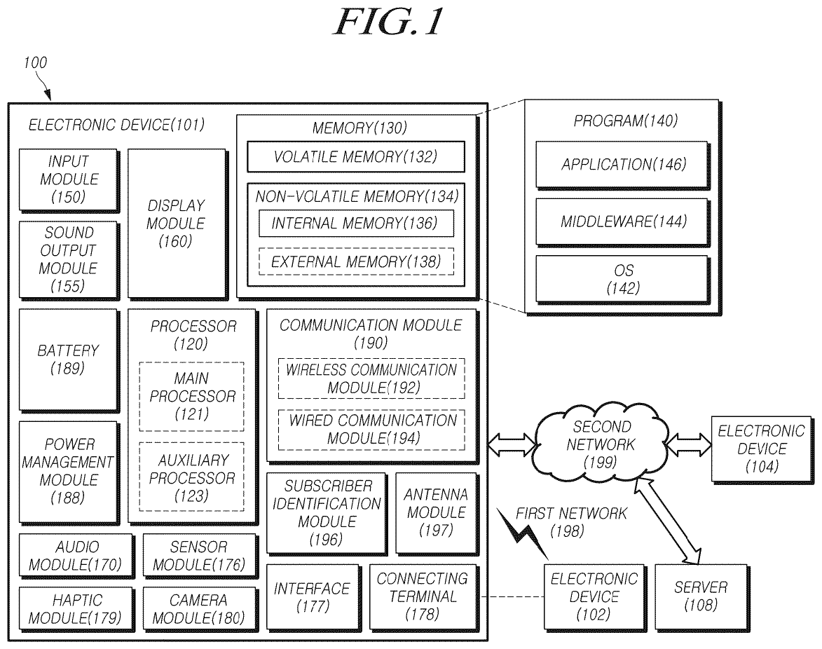

FIG. 1 is a block diagram illustrating an example electronic device in a network environment according to an embodiment.

In FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In an embodiment, at least one (e.g., the connecting terminal 178) of the components may be omitted from the electronic device 101, or one or more other components may be added in the electronic device 101. According to an embodiment, some (e.g., the sensor module 176, the camera module 180, or the antenna module 197) of the components may be integrated into a single component (e.g., the display module 160).

The processor 120 may execute, for example, software (e.g., a program 140) to control at least one other component (e.g., a hardware or software component) of the electronic device 101 coupled with the processor 120 and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134.

The number of processors 120 may be one or more. For example, the processor 120 may have a structure of a multi-core processor such as dual core, quad core, or hexa core.

The processor 120 may control the operations of the electronic device 101 by executing the instructions stored in the memory 130. For example, the processor 120 may correspond to a plurality of processors that divide a plurality of operations between processors and collectively perform the operations.

According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, in case that the electronic device 101 includes the main processor 121 and the auxiliary processor 123, the auxiliary processor 123 may be configured to use lower power than the main processor 121 or to be specified for a designated function. The auxiliary processor 123 may be implemented as separate from, or as part of the main processor 121. The processor 120 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, at least one processor may include a combination of processors performing various of the recited/disclosed functions, e.g., in a distributed manner. At least one processor may execute program instructions to achieve or perform various functions.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. The artificial intelligence model may be generated via machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by other component (e.g., the processor 120) of the electronic device 101, from the outside (e.g., a user) of the electronic device 101. The input module 150 may include, for example, a microphone, a mouse, a keyboard, keys (e.g., buttons), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor configured to detect a touch, or a pressure sensor configured to measure the intensity of a force generated by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an accelerometer, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high-definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or motion) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wiredly) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wiredly) communication or a wireless communication. According to an embodiment, the communication module 190 may include a communication module 192 (e.g., a cellular communication module, a short-range communication module, or a global navigation satellite system (GNSS) communication module) or a wired communication module 194 (e.g., a local area network (LAN) communication module or a power line communication (PLC) module). A corresponding one of these communication modules may communicate with the external electronic device 104 via a first network 198 (e.g., a short-range communication network, such as Bluetooth™, wireless-fidelity (Wi-Fi) direct, or infrared data association (IrDA)) or a second network 199 (e.g., a long-range communication network, such as a legacy cellular network, a 5G network, a next-generation communication network, the Internet, or a computer network (e.g., local area network (LAN) or wide area network (WAN)). These various types of communication modules may be implemented as a single component (e.g., a single chip), or may be implemented as multi components (e.g., multi chips) separate from each other. The communication module 192 may identify or authenticate the electronic device 101 in a communication network, such as the first network 198 or the second network 199, using subscriber information (e.g., international mobile subscriber identity (IMSI)) stored in the subscriber identification module 196.

The communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The communication module 192 may support a high-frequency band (e.g., the mmWave band) to achieve, e.g., a high data transmission rate. The communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The communication module 192 may support various requirements specified in the electronic device 101, an external electronic device (e.g., the electronic device 104), or a network system (e.g., the second network 199). According to an embodiment, the communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device). According to an embodiment, the antenna module 197 may include one antenna including a radiator formed of a conductor or conductive pattern formed on a substrate (e.g., a printed circuit board (PCB)). According to an embodiment, the antenna module 197 may include a plurality of antennas (e.g., an antenna array). In this case, at least one antenna appropriate for a communication scheme used in a communication network, such as the first network 198 or the second network 199, may be selected from the plurality of antennas by, e.g., the communication module 190. The signal or the power may then be transmitted or received between the communication module 190 and the external electronic device via the selected at least one antenna. According to an embodiment, other parts (e.g., radio frequency integrated circuit (RFIC)) than the radiator may be further formed as part of the antenna module 197.

According to various embodiments, the antenna module 197 may form a mmWave antenna module. According to an embodiment, the mm Wave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface, or mobile industry processor interface (MIPI)).

According to an embodiment, instructions or data may be transmitted or received between the electronic device 101 and the external electronic device 104 via the server 108 coupled with the second network 199. The external electronic devices 102 or 104 each may be a device of the same or a different type from the electronic device 101. According to an embodiment, all or some of operations to be executed at the electronic device 101 may be executed at one or more of the external electronic devices 102, 104, or 108. For example, in case that the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request and transfer an outcome of the performing to the electronic device 101. The electronic device 101 may provide the outcome, with or without further processing of the outcome, as at least part of a reply to the request. To that end, cloud computing, distributed computing, mobile edge computing (MEC), or client-server computing technology may be used, for example. The electronic device 101 may provide ultra low-latency services using, e.g., distributed computing or mobile edge computing. In an embodiment, the external electronic device 104 may include an Internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or health-care) based on 5G communication technology or IoT-related technology.

The electronic device according to various embodiments of the disclosure may be one of various types of electronic devices. The electronic devices may include, for example, a portable communication device (e.g., a smartphone), a computer device, a portable multimedia device, a portable medical device, a camera, a wearable device, a home appliance, or the like. According to embodiments of the disclosure, the electronic device is not limited to the devices described above.

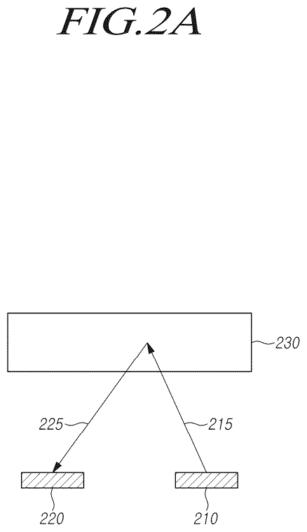

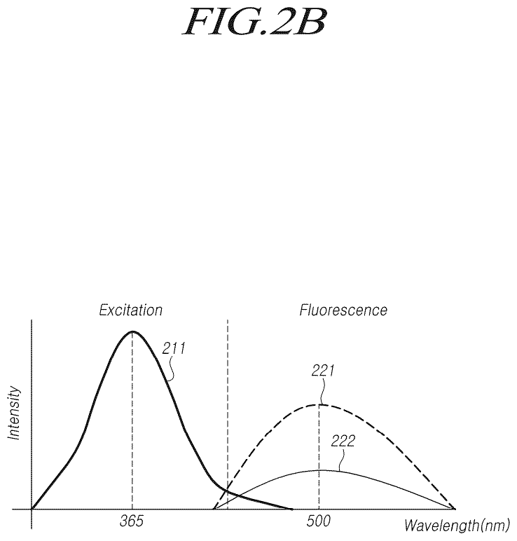

FIGS. 2A and 2B include a diagram and graph illustrating an example method of obtaining biometric information using an optical sensor, according to an embodiment.

In FIG. 2A, an electronic device (e.g., the electronic device 101 of FIG. 1) may include an optical sensor. The optical sensor may include a light emitting unit 210 including a light emitting diode (LED) or laser that emits light 215, and a light receiving unit 220 including a photodiode (PD) that receives light 225. Biometric information may be obtained by emitting light 215 to skin 230 using the light emitting unit 210 and then measuring light 225 that returns after reacting with substances or blood containers in skin tissue using the light receiving unit 220. Although not illustrated in FIG. 2A, at least a portion of the light 215 emitted from the light emitting unit 210 may penetrate the skin 230. For example, advanced glycation end-products (AGEs) in skin 230 may react with ultraviolet (UV) light emitted from the light emitting unit 210 to generate a fluorescence signal. The AGEs may be detected by measuring a fluorescence signal using the light receiving unit 220. The AGEs are substances formed in case that proteins or lipids are exposed to sugar and become glycated. For example, AGEs may include N6-(1-carboxyethyl)lysine, N6-(carboxymethyl)lysine, N5-(5-hydro-5-methyl-4-imidazolon-2-yl)-ornithine, and/or pentosidine. The AGEs may accumulate in various portions of the human body (e.g., organs, muscles, joints, blood containers, skin) and may be used as biomarkers for aging and various physical diseases such as cancer, cardiovascular disease, or diabetes.

In an embodiment, the graph of FIG. 2B illustrates on the vertical axis the intensity of light 211 emitted from the light emitting unit 210 and fluorescence 221 or 222 received by the light receiving unit 220 along the horizontal axis of wavelength. The light 211 emitted from the light emitting unit 210 may react with an object to generate fluorescence 221 or 222. The light receiving unit 220 may receive the fluorescence 221 or 222. In case that the object includes many fluorescent substances, the intensity of the fluorescence 221 may be high. In case that the object includes few fluorescent substances, the intensity of the fluorescence 222 may be low. The amount of fluorescent substances in the object may be measured by irradiating the object with light 211 and analyzing the intensity of fluorescence 221 or 222 emitted from the object.

For example, a peak wavelength or center wavelength of the light 211 emitted by the light emitting unit 210 may be included in a range of about 360 nm to 370 nm. AGEs may emit fluorescence 221 or 222 having a peak wavelength of about 500 nm in response to emission of the light 211. In case that there are many AGEs in the body, the intensity of the fluorescence 221 may be high. In case that there are few AGEs in the body, the intensity of the fluorescence 222 may be low. The electronic device 101 may measure the amount of AGEs in the body by irradiating the body with light 211 and analyzing the intensity of fluorescence 221 or 222 emitted from the body.

In an embodiment, contact between an object and an optical sensor is desired (e.g., necessary) to enhance the accuracy of fluorescence 221 or 222 measurement. For example, in case of measuring AGEs in the body, the sensor and the body is substantially in contact to accurately capture fluorescence caused by AGEs. Fluorescence measurement may depend on whether the sensor contacts an object and/or on a distance between the sensor and the object, so consistent (e.g., uniform) contact should be made to obtain reliable data. In case that the sensor and the object are in contact, external lighting interference may be minimized and/or reduced, so the accuracy of fluorescence measurement may be enhanced.

In an embodiment, contact may refer, for example, to physical contact. Contact between the electronic device 101 and an object may refer, for example, to a distance between the surface of a measurement sensor included in the electronic device 101 and the surface of the object corresponding to about 0 mm. Even in case that the surface of the measurement sensor protrudes convexly and/or in case that the measurement sensor is pressed against the object, the distance between the measurement sensor and the object is about 0 mm in case of a contact state. In this case, the contact pressure has increased. In a contact state, some or all of the elements of the optical sensor (e.g., the light emitting unit 210 and the light receiving unit 220) may contact the object.

In an embodiment, the electronic device 101 may determine a contact state using a displacement sensor (laser type or resistive type) that measures a distance between the sensor and the object and/or a bioelectrode. For example, in case that bioelectrodes contact skin, the impedance value across the electrodes may change, so the electronic device 101 may determine a contact state using the bioelectrode. On the other hand, in case that the surface of the bioelectrode is contaminated by sweat or foreign objects, there is a possibility of incorrectly recognizing a contact state even though the electronic device 101 does not contact the body.

In an embodiment, the electronic device 101 may be a wearable electronic device, and the electronic device 101 may determine whether the electronic device 101 is worn on a body. The electronic device 101 may include an optical sensor. The electronic device 101 may determine whether the electronic device 101 is worn on a body by emitting light using an optical sensor and detecting light that returns due to actions such as reflection. While most objects absorb light in the infrared wavelength band, a body may significantly reflect light in the infrared wavelength band. The electronic device 101 may determine whether the electronic device 101 is worn on a body using light in the infrared wavelength band. The electronic device 101 may drive a biometric sensor to obtain biometric information (e.g., detect a target substance in the body) based on determining that the electronic device 101 is worn on a body. The electronic device 101 may determine that the electronic device 101 is worn on a body even in case that the electronic device 101 and the body are not in contact. Even in case that the electronic device 101 does not contact a body, it may fall within the criteria for determining whether the electronic device 101 is worn on a body. However, in case of measuring fluorescent substances included in an object, there is a need to determine whether the electronic device 101 contacts the object.

In an embodiment, the disclosure may provide a method of detecting whether a sensor contacts an object. In an embodiment, the disclosure may provide a method of detecting whether a sensor contacts a body in case of measuring AGEs in the body. In an embodiment, the disclosure may provide an accurate and reliable contact detection method using a pair of optical signals obtained for UV incident light.

In an embodiment, the contact detection method may be utilized not only for AGEs but also for detecting various biometric information. Biometric information may include, e.g., blood pressure, body temperature, blood glucose, genetic material (e.g., deoxyribonucleic acid (DNA), ribonucleic acid (RNA)), proteins, bacteria, and/or viruses. Accurate and stable body contact may be required in case of detecting biometric information. The contact detection method may enhance the accuracy of biometric information measurement by recognizing circumstances where body contact is lost due to changes in wearing state (e.g., loose straps) and environments (e.g., sleep and daily activities).

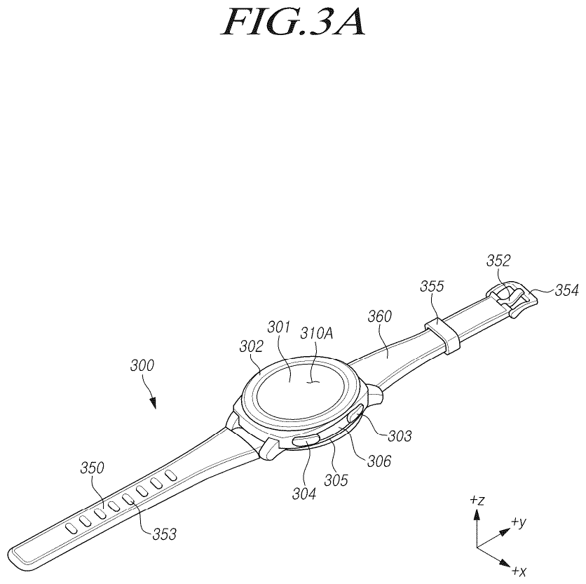

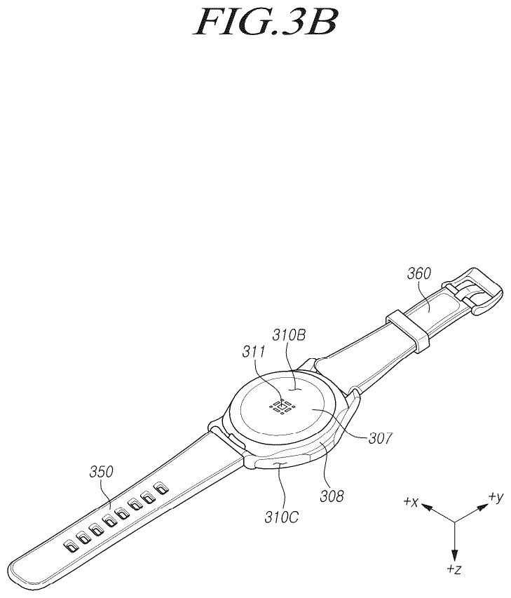

FIG. 3A is a front perspective view illustrating a front surface of an example electronic device (e.g., the electronic device 101 of FIG. 1) 300 according to an embodiment, and FIG. 3B is a rear perspective view illustrating a rear surface of the electronic device 300 according to an embodiment.

In FIGS. 3A and 3B, the electronic device 300 according to an embodiment may include a housing 310 including a first side (or front side (e.g., front surface)) 310A, a second side (or rear side (e.g., rear surface)) 310B, and a lateral side (e.g., lateral surface) 310C surrounding the space between the first side 310A and the second side 310B, and attachment members 350 and 360 connected to at least a portion of the housing 310 and configured to detachably fasten the electronic device 300 to a portion of a body of a user (e.g., wrist, ankle). In an embodiment (not illustrated), a housing may also refer to a structure forming portions of the first side 310A, the second side 310B, and the lateral side 310C of FIGS. 3A and 3B. According to an embodiment, the first side 310A may be formed by a front plate 301 (e.g., a glass plate including various coating layers, or a polymer plate) that is at least partially substantially transparent. The second side 310B may be formed of a substantially opaque rear plate 307. The rear plate 307 may be formed of, e.g., laminated or colored glass, ceramic, polymer, metal (e.g., aluminum, stainless steel (STS), or magnesium), or a combination of at least two thereof. The lateral side 310C may be formed by a side bezel structure (or “side member”) 306 that combines with the front plate 301 and the rear plate 307 and includes metal and/or polymer. According to an embodiment, the rear plate 307 and the side bezel plate 306 may be integrally formed together and include the same material (e.g., a metal, such as aluminum). The attachment members 350 and 360 may be formed of various materials in various shapes. They may be formed integrally or as a plurality of unit links movable relative to each other by woven fabric, leather, rubber, urethane, metal, ceramic, or a combination of at least two of the materials.

According to an embodiment, the electronic device 300 may include at least one or more of a display (not illustrated), an audio module 305, 308, a sensor module 311, and a key input device 302, 303, 304. In an embodiment, the electronic device 300 may omit at least one of the components (e.g., the key input device 302, 303, 304) or may additionally include other components.

A display may, e.g., be visible through a substantial portion of the front plate 301. The display may have a shape corresponding to the shape of the front plate 301, e.g., a circle, ellipse, or polygon. The display may be coupled with, or disposed adjacent, a touch detection circuit, a pressure sensor capable of measuring the strength (pressure) of touches, and/or fingerprint sensor.

In an embodiment, the audio module 305, 308 may include a microphone hole 305 and a speaker hole 308. The microphone hole 305 may have a microphone disposed therein to obtain external sound, and in an embodiment, multiple microphones may be disposed to detect the direction of sound. The speaker hole 308 may be used for an external speaker or a receiver for phone talks. In an embodiment, the speaker hole 308 and the microphone hole 305 may be implemented as a single hole, or a speaker may be included without the speaker hole 308 (e.g., a piezo speaker).

In an embodiment, the sensor module 311 may generate an electrical signal or data value corresponding to the internal operation state of the electronic device 300 or the external environmental state. The sensor module 311 may include, e.g., a biometric sensor module 311 (e.g., a heart rate monitor sensor) disposed on the second side 310B of the housing 310. The electronic device 300 may further include at least one of sensor modules not illustrated, e.g., a gesture sensor, a gyro sensor, a barometric pressure sensor, a magnetic sensor, an acceleration sensor, a grip sensor, a color sensor, an optical sensor, an infrared (IR) sensor, a biometric sensor, a temperature sensor, a humidity sensor, or an illuminance sensor.

In an embodiment, the key input device 302, 303, 304 may include a wheel key 302 disposed on the first side 310A of the housing 310 and rotatable in at least one direction, and/or side key buttons 302, 303 disposed on the lateral side 310C of the housing 310. The wheel key 302 may have a shape corresponding to the shape of the front plate 301. In an embodiment, the electronic device 300 may not include some or all of the above-described key input devices 302, 303, 304, and the key input devices 302, 303, 304 not included may be implemented in other forms such as soft keys on the display.

In an embodiment, the attachment members 350, 360 may be detachably attached to at least a partial area of the housing 310. The attachment members 350, 360 may include one or more of a fixing member 352, a fixing member fastening hole 353, a band guide member 354, and a band fixing loop 355.

In an embodiment, the fixing member 352 may be configured to fix the housing 310 and the attachment members 350, 360 to a portion of a user's body (e.g., wrist, ankle). The fixing member fastening holes 353 may fix the housing 310 and the attachment members 350 and 360 to the user's body portion, corresponding to the fixing member 352. The band guide member 354 may be configured to restrict movement of the fixing member 352 to a certain range in case that the fixing member 352 fits into one of the fixing member fastening holes 353, thereby allowing the attachment members 350 and 360 to be tightly (e.g., firmly) bound (e.g., connected or attached) onto the user's body portion. The band fixing loop 355 may limit the range of movement of the attachment members 350, 360 in case that the fixing member 352 and the fixing member fastening hole 353 are fastened.

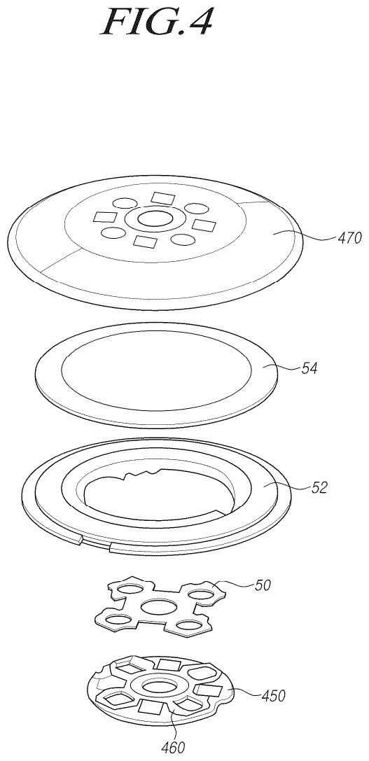

FIG. 4 is an exploded perspective view illustrating an example in which an optical sensor is disposed in an electronic device according to an embodiment.

In FIG. 4, an optical sensor according to an embodiment may be included in the sensor module 311 of FIG. 3B. The optical sensor may include a light emitting element and a light receiving element. According to an embodiment, the light emitting element and the light receiving element may respectively include a plurality of light emitting elements and a plurality of light receiving elements. The plurality of light emitting elements may include a plurality of LEDs or lasers corresponding to a plurality of wavelength bands, and the plurality of light receiving elements may include a plurality of PDs corresponding to a plurality of wavelength bands. For example, the optical sensor may include a plurality of LEDs or lasers that emit at least one light among UV light, violet light, blue light, green light, yellow light, red light, or IR light, and a plurality of PDs that receive at least one light among UV light, violet light, blue light, green light, yellow light, orange light, red light, or IR light.

According to an embodiment, the optical sensor may be a biometric sensor that emits light toward a body and receives light generated as a result of an interaction between the emitted light and the body. The biometric sensor may include, but is not limited to, a biomarker sensor for detecting specific substances or components in the body. For example, the biometric sensor may include a photoplethysmogram (PPG) sensor, a heart rate sensor, a heart rate variability sensor, a saturation of partial pressure oxygen (SpO₂) sensor, and/or a blood pressure sensor. A biomarker is an indicator of changes inside the body such as cells, blood containers, proteins, DNA, RNA, and metabolites inside the body, and may detect AGEs, blood glucose, alcohol, and/or antioxidants.

The optical sensor may include a light emitter and a light receiver. The light emitter may include at least one light emitting element. The light receiver may include at least one light receiving element. For example, the light emitting element may include an LED, a laser, and/or a vertical cavity surface emitting laser, but is not limited thereto. For example, the light receiving element may include a PD and/or a complementary metal oxide semiconductor sensor, but is not limited thereto. The light receiver may receive light reflected or transmitted from light emitted from the light emitter and transmit a value converted through an analog to digital converter to a memory or sensor buffer. The light receiver may include an optical filter for passing or filtering out light of a specific band.

According to an embodiment, the optical sensor may include a plurality of light emitting elements and a plurality of light receiving elements disposed on a circuit board 450, and a barrier member 460 may be disposed on the circuit board 450. Further, the circuit board 450 may be disposed within the electronic device 400 such that one side (e.g., surface) of the circuit board 450 on which the plurality of light emitting elements and the plurality of light receiving elements are disposed faces rear glass 470 of the electronic device 400. The barrier member 460 may be implemented with an opaque material that does not transmit light. In case that the circuit board 450 is viewed vertically (e.g., in a direction perpendicular to the glass 470 and the circuit board 450), the barrier member 460 may not overlap the plurality of light emitting elements and the plurality of light receiving elements on the circuit board 450.

According to an embodiment, the barrier member 460 disposed on one side (e.g., surface) of the circuit board 450 may be attached to the rear glass 470 through an adhesive member 50. The barrier member 460 disposed on one side (e.g., surface) of the circuit board 450 may be attached to the rear glass 470 such that the optical sensor disposed on one side (e.g., surface) of the circuit board 450 corresponds to an area in the rear glass 470 through which light passes. According to an embodiment, the adhesive member 50 may be implemented with an opaque material that does not allow light to pass through.

According to an embodiment, a member 52 including an antenna coil may be attached to the rear glass 470 through an adhesive member 54. In this case, the opening of the member 52 including the antenna coil and the opening of the adhesive member 54 may be formed to be larger than one side (e.g., surface) of the circuit board 450, and accordingly, the barrier member 460 of the circuit board 450 may be attached to the rear glass 470.

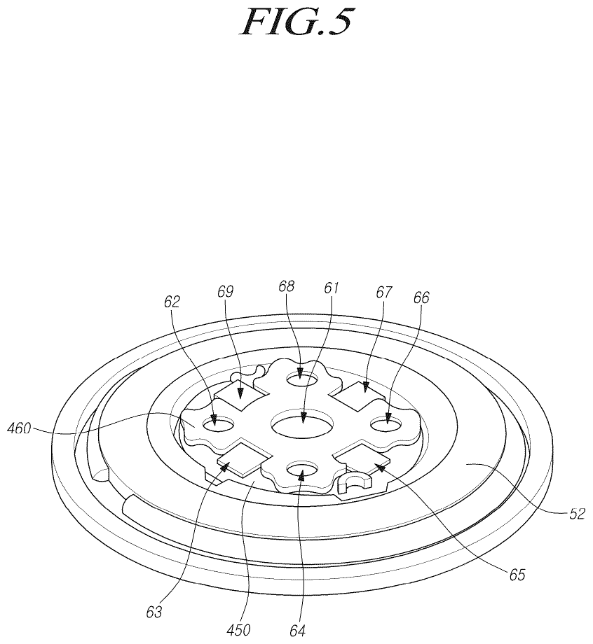

FIG. 5 is a perspective view illustrating various regions on a circuit board divided by a barrier member disposed on the circuit board, according to an embodiment.

In FIG. 5, a barrier member 460 according to an embodiment may be disposed on a circuit board 450, and an area on one surface of the circuit board 450 may be divided into a plurality of regions 61, 62, 63, 64, 65, 66, 67, 68, 69 by the barrier member 460. The plurality of regions 61, 62, 63, 64, 65, 66, 67, 68, 69 according to an embodiment may be spatially separated from each other on the circuit board 450.

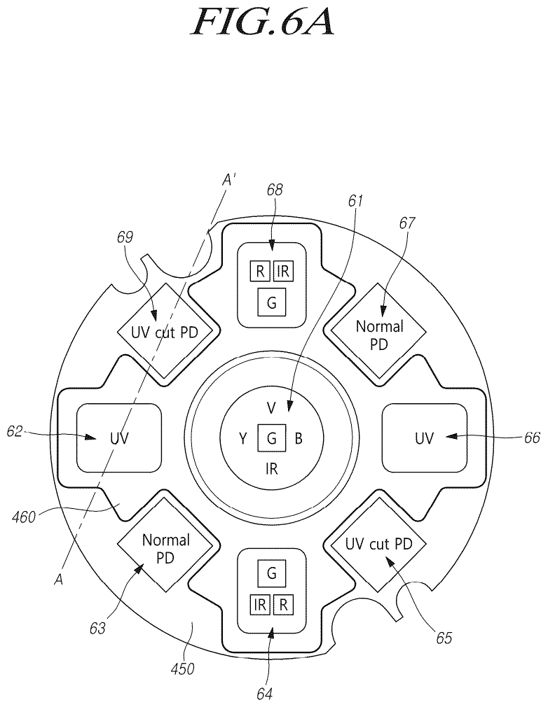

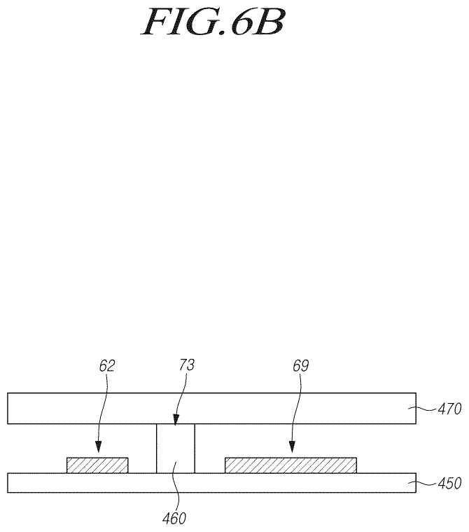

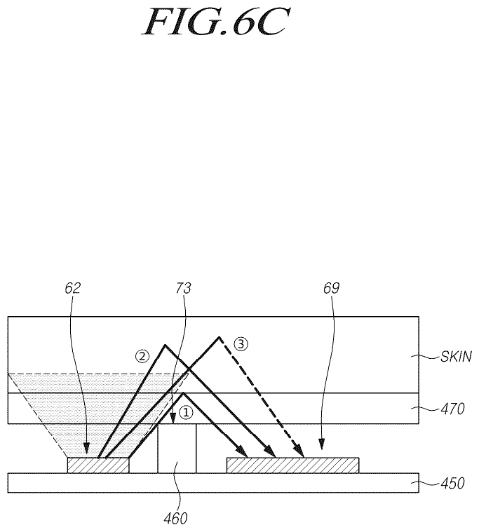

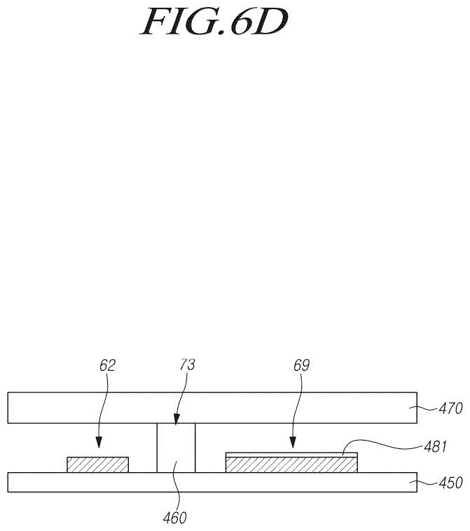

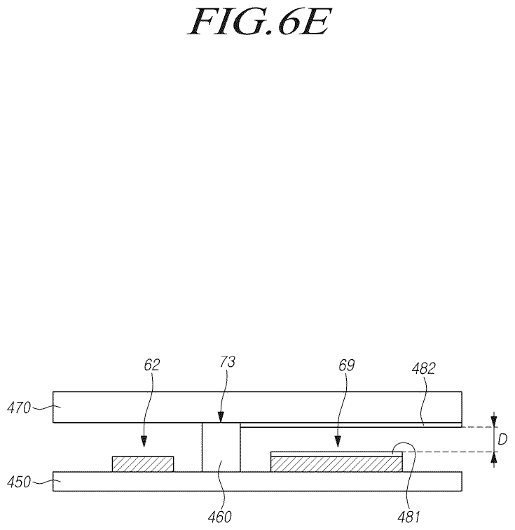

According to an embodiment, the rear glass (470 of FIG. 4) may include a plurality of windows corresponding to the plurality of regions 61, 62, 63, 64, 65, 66, 67, 68, 69 on the circuit board 450. The plurality of windows may have different shapes according to the type of element disposed in the corresponding regions 61, 62, 63, 64, 65, 66, 67, 68, 69. For example, windows corresponding to the regions 61, 62, 64, 66, 68 where light emitting elements are disposed may have a circular shape. For example, windows corresponding to the regions 63, 65, 67, 69 where light receiving elements are disposed may have a rectangular shape. The plurality of windows may be transparent but are not limited thereto and may be opaque or translucent. According to an embodiment, at least one window includes an optical filter for passing or blocking light of a specific band and may have a specific color. For example, at least one window corresponding to a light receiving element may include an optical filter for blocking the UV light and may be green. An optical filter for passing or blocking light of a specific band is described in greater detail below with reference to FIGS. 6D and 6E.

The plurality of regions 61, 62, 63, 64, 65, 66, 67, 68, 69, according to an embodiment, may include, e.g., a first region 61, a second region 62, a third region 63, a fourth region 64, a fifth region 65, a sixth region 66, a seventh region 67, an eighth region 68, and a ninth region 69.

For example, the first region 61 may be positioned in a central portion of the circuit board 450. Further, the second region 62, the third region 63, the fourth region 64, the fifth region 65, the sixth region 66, the seventh region 67, the eighth region 68, and the ninth region 69 may surround the first region 61 and be positioned along the edge of the circuit board 450.

According to an embodiment, some of the plurality of regions 61, 62, 63, 64, 65, 66, 67, 68, 69 may be formed by openings of the barrier member 460, and the remaining portions may be formed by concave (e.g., concave-shaped) portions at the edge of the barrier member 460. For example, the first region 61, the second region 62, the fourth region 64, the sixth region 66, and the eighth region 68 may be formed by a plurality of openings of the barrier member 460. For example, the third region 63, the fifth region 65, the seventh region 67, and the ninth region 69 may be formed by portions having a concave shape toward the center of the barrier member 460 from the edge of the barrier member 460. Accordingly, even in case that the size of the portion attached to the barrier member 460 on one side (e.g., surface) of the circuit board 450 decreases, the plurality of regions 61, 62, 63, 64, 65, 66, 67, 68, 69 on one side (e.g., surface) of the circuit board 450 may be spatially separated from each other.

The barrier member 460 according to an embodiment may be implemented with an opaque material through which light does not pass through. For example, the barrier member 460 may have a black color and may include at least one material among polycarbonate, silicone, acrylonitrile butadiene styrene (ABS), polytetrafluoroethylene (PTFE), Teflon, polyamide, epoxy, thermoplastic polyurethane (TPU), or metal, but the disclosure is not limited thereto.

In the plurality of regions 61, 62, 63, 64, 65, 66, 67, 68, 69, according to an embodiment, e.g., at least some of the plurality of light emitting elements and the plurality of light receiving elements included in the optical sensor may be disposed. The elements disposed in the plurality of regions 61, 62, 63, 64, 65, 66, 67, 68, 69, according to an embodiment, is described in more detail in FIG. 6A.

FIG. 6A is a diagram illustrating various elements of an optical sensor disposed in a plurality of regions divided by a barrier member, according to an embodiment.

In FIG. 6A, at least some of the plurality of light emitting elements and the plurality of light receiving elements included in the optical sensor may be disposed in the plurality of regions 61, 62, 63, 64, 65, 66, 67, 68, 69 according to an embodiment.

According to an embodiment, a light emitting element may be disposed in the first region 61. For example, at least one of a red light emitting element, a green light emitting element, a blue light emitting element, a violet light emitting element, a yellow light emitting element, or an IR light emitting element may be disposed in the first region 61. For example, as illustrated in FIG. 6A, a green light emitting element, a blue light emitting element, a violet light emitting element, a yellow light emitting element, and an IR light emitting element may be disposed in the first region 61. According to an embodiment, the red light emitting element may be an LED or laser that emits light having a wavelength within a range of about 620 nm to 720 nm, but the disclosure is not limited thereto. For example, the red light emitting element may be an LED or laser that emits light having a center wavelength of about 660 nm. According to an embodiment, the yellow light emitting element may be an LED or laser that emits light having a wavelength within a range of about 570 nm to 590 nm, but the disclosure is not limited thereto. For example, the yellow light emitting element may be an LED or laser that emits light having a center wavelength of about 580 nm. According to an embodiment, the green light emitting element may be an LED or laser that emits light having a wavelength within a range of about 495 nm to 570 nm, but the disclosure is not limited thereto. For example, the green light emitting element may be an LED or laser that emits light having a center wavelength of about 525 nm. According to an embodiment, the violet light emitting element may be an LED or laser that emits light having a wavelength within a range of about 385 nm to 430 nm, but the disclosure is not limited thereto. For example, the violet light emitting element may be an LED or laser that emits light having a center wavelength of about 405 nm. According to an embodiment, the blue light emitting element may be an LED or laser that emits light having a wavelength within a range of about 450 nm to 495 nm, but the disclosure is not limited thereto. For example, the blue light emitting element may be an LED or laser that emits light having a center wavelength of about 470 nm. According to an embodiment, the IR light emitting element may be an LED or laser that emits light having a wavelength within a range of about 700 nm or more, but the disclosure is not limited thereto. For example, the IR light emitting element may be an LED or laser that emits light having a center wavelength of about 940 nm.

According to an embodiment, a light emitting element may be disposed in the second region 62. For example, as illustrated in FIG. 6A, a UV light emitting element may be disposed in the second region 62, and the UV light emitting element may be spatially separated from other elements (e.g., a red light emitting element, a green light emitting element, a blue light emitting element, a violet light emitting element, a yellow light emitting element, an IR light emitting element, Normal PD, and UV cut PD) by the barrier member 460. For example, the UV light emitting element may include an LED or laser that emits light having a wavelength within a range of about 100 nm to 400 nm, but the disclosure is not limited thereto. For example, the UV light emitting element may include an LED or laser that emits light having a center wavelength of about 365 nm.

According to an embodiment, a light receiving element may be disposed in the third region 63. For example, as illustrated in FIG. 6A, a Normal PD may be disposed in the third region 63. The Normal PD may be, e.g., a light receiving element for receiving light having a wavelength within a range of about 340 nm to 980 nm.

According to an embodiment, a light emitting element may be disposed in the fourth region 64. For example, at least one of a red light emitting element, a green light emitting element, a blue light emitting element, a violet light emitting element, a yellow light emitting element, or an IR light emitting element may be disposed in the fourth region 64. For example, as illustrated in FIG. 6A, a green light emitting element, a red light emitting element, and an IR light emitting element may be disposed in the fourth region 64. According to an embodiment, the red light emitting element may be a light emitting element that emits light having a wavelength within a range of about 620 nm to 720 nm, and may be, e.g., an LED or laser that emits light having a center wavelength of about 660 nm. According to an embodiment, the green light emitting element may be an LED or laser that emits light having a wavelength within a range of about 495 nm to 570 nm, and may be, e.g., an LED or laser that emits light having a center wavelength of about 525 nm. According to an embodiment, the IR light emitting element may be an LED or laser that emits light having a wavelength within a range of about 700 nm or more, and may be, e.g., an LED or laser that emits light having a center wavelength of about 940 nm.

According to an embodiment, a light receiving element may be disposed in the fifth region 65. For example, as illustrated in FIG. 6A, a UV cut PD may be disposed in the fifth region 65. The UV cut PD may receive light of a wavelength excluding at least the UV wavelength band, for example. The UV cut PD may include a light-filtering member that blocks light in the UV wavelength band. For example, the UV cut PD may receive light having a wavelength within a range of about 480 nm to 980 nm, but the disclosure is not limited thereto. A light-filtering member for passing or blocking light of a specific band is further described with reference to FIGS. 6D and 6E.

According to an embodiment, a light emitting element may be disposed in the sixth region 66 at a position substantially opposite to the second region 62 based on the center of the circuit board 450. For example, as illustrated in FIG. 6A, a UV light emitting element may be disposed in the sixth region 66. According to an embodiment, the UV light emitting element may be spatially separated from other elements (e.g., a red light emitting element, a green light emitting element, a blue light emitting element, a violet light emitting element, a yellow light emitting element, an IR light emitting element, Normal PD, and UV cut PD) by the barrier member 460. For example, the UV light emitting element may include an LED or laser that emits light having a wavelength within a range of about 100 nm to 400 nm, but the disclosure is not limited thereto. For example, the UV light emitting element may include an LED or laser that emits light having a center wavelength of about 365 nm.

According to an embodiment, a light receiving element may be disposed in the seventh region 67 at a position substantially opposite to the third region 63 based on the center of the circuit board 450. For example, as illustrated in FIG. 6A, a Normal PD may be disposed in the seventh region 67. The Normal PD may be, e.g., a light receiving element for receiving light having a wavelength within a range of about 340 nm to 980 nm.

According to an embodiment, a light emitting element may be disposed in the eighth region 68 at a position substantially opposite to the fourth region 64 based on the center of the circuit board 450. For example, at least one of a red light emitting element, a green light emitting element, a blue light emitting element, a violet light emitting element, a yellow light emitting element, or an IR light emitting element may be disposed in the eighth region 68. For example, as illustrated in FIG. 6A, a red light emitting element, a green light emitting element, and an IR light emitting element may be disposed in the eighth region 68. According to an embodiment, the red light emitting element may be a light emitting element that emits light having a wavelength within a range of about 620 nm to 720 nm, and may be, e.g., an LED or laser that emits light having a center wavelength of about 660 nm. According to an embodiment, the green light emitting element may be a light emitting element that emits light having a wavelength within a range of about 495 nm to 570 nm, and may be, e.g., an LED or laser that emits light having a center wavelength of about 525 nm. According to an embodiment, the IR light emitting element may be a light emitting element that emits light having a wavelength within a range of about 700 nm or more, and may be, e.g., an LED or laser that emits light having a center wavelength of about 940 nm.

According to an embodiment, a light receiving element may be disposed in the ninth region 69 at a position substantially opposite to the fifth region 65 based on the center of the circuit board 450. For example, as illustrated in FIG. 6A, a UV cut PD may be disposed in the ninth region 69. The UV cut PD may receive light of a wavelength excluding at least the UV wavelength band, for example. The UV cut PD may include a light-filtering member that blocks light in the UV wavelength band. For example, the UV cut PD may receive light having a wavelength within a range of about 480 nm to 980 nm, but the disclosure is not limited thereto. A light-filtering member for passing or blocking light of a specific band is further described in greater detail below with reference to FIGS. 6D and 6E.

According to an embodiment, the electronic device 400 may emit UV light toward skin of a user and then measure a fluorescence signal generated by the emitted UV light reacting with AGEs in the skin of the user.

For example, the electronic device 400 may emit UV light toward a body of a user (e.g., skin) by controlling at least one of the light emitting element (e.g., UV light emitting element) in the first region 61 and the light emitting element (e.g., UV light emitting element) in the sixth region 66. Further, AGEs in the body of the user may emit a fluorescence signal in response to UV light. According to an embodiment, the electronic device 400 may receive a fluorescence signal emitted from the body of the user using at least one of the normal PDs disposed in the third region 63 and the seventh region 67 of the circuit board 450, and the UV cut PDs disposed in the fifth region 65 and the ninth region 69. Thereafter, the electronic device 400 may emit light of a predetermined wavelength toward the body of the user (e.g., skin) by controlling at least one of the red light emitting element, the green light emitting element, the blue light emitting element, the violet light emitting element, the yellow light emitting element, or the IR light emitting element disposed on the circuit board 450. Further, the electronic device 400 may receive reflected light of the emitted light reflected by the body of the user using at least one of the normal PDs disposed in the third region 63 and the seventh region 67 of the circuit board 450, and the UV cut PDs disposed in the fifth region 65 and the ninth region 69, and may correct the fluorescence signal considering a skin color of the user based on the received reflected light. The electronic device 400 may estimate the AGEs of the user based on the corrected fluorescence signal. For example, light having a peak wavelength of about 365 nm (UV) is emitted to skin, and AGEs in the skin react to that light to generate a fluorescence signal in a wavelength band of about 380 nm to 600 nm centered around about 500 nm. The amount of AGEs in the body may be estimated by analyzing the magnitude of this fluorescence signal.

According to an embodiment, in order to more accurately estimate AGEs, signals other than the fluorescence signal generated from skin should be more accurately separated or controlled (e.g., restricted or suppressed). For example, in case that fluorescence is generated from the red light emitting element, the blue light emitting element, the green light emitting element, the violet light emitting element, the yellow light emitting element, and/or the IR light emitting element due to UV light, the fluorescence generated from the red light emitting element, the blue light emitting element, the green light emitting element, and/or the IR light emitting element may act as noise or crosstalk for the estimation of the AGEs. Accordingly, according to an embodiment, in order to accurately estimate the AGEs, the elements of the optical sensor in the electronic device 400 may be disposed to be spatially separated by the barrier member 460 on the circuit board 450.

FIG. 6B is a cross-sectional view illustrating a plurality of regions divided by a barrier member, according to an embodiment. FIG. 6B illustrates the A-A′ cross-section of FIG. 6A. In FIG. 6B, the second region 62 and the ninth region 69 may be spatially separated from each other by the barrier member 460 according to an embodiment. The barrier member 460 is disposed on the circuit board 450, and the second region 62 and the ninth region 69 may be spatially separated from each other by the disposed barrier member 460.

In an embodiment, the barrier member 460 disposed on the circuit board 450 surrounds the light emitting elements of one region, so that the regions 61, 62, 63, 64, 65, 66, 67, 68, 69 where the light emitting elements or light receiving elements are disposed may be spatially separated from each other. For example, the barrier member 460 disposed on the circuit board 450 may surround the light emitting elements respectively disposed in the first region 61, the second region 62, the fourth region 64, the sixth region 66, and the eighth region 68, whereby the regions 61, 62, 63, 64, 65, 66, 67, 68, 69 where the light emitting elements or light receiving elements are disposed may be spatially separated from each other. By the disposed barrier member 460, the elements disposed in each region 61, 62, 63, 64, 65, 66, 67, 68, 69 may be isolated by region. The barrier member 460 may surround the sides of the elements disposed on one side (e.g., surface) of the circuit board 450 and may not cover the upper sides (e.g., surfaces) of the elements so that the light emitting elements may emit light through the glass 470 and the light receiving elements may receive light through the glass 470.

According to an embodiment, the rear glass 470 may include a plurality of windows corresponding to the plurality of regions 61, 62, 63, 64, 65, 66, 67, 68, 69 of the circuit board 450. The plurality of windows may have different shapes according to the type of element disposed in the corresponding regions 61, 62, 63, 64, 65, 66, 67, 68, 69. For example, windows corresponding to the regions 61, 62, 64, 66, 68 where light emitting elements are disposed may have a circular shape. For example, windows corresponding to the regions 63, 65, 67, 69 where light receiving elements are disposed may have a rectangular shape. The plurality of windows may be transparent but are not limited thereto and may be opaque or translucent. According to an embodiment, at least one window includes a light-filtering member for passing or blocking light of a specific band and may have a specific color. For example, at least one window corresponding to a light receiving element may include a light-filtering member for blocking UV light and may be green. A light-filtering member for passing or blocking light of a specific band is described in greater detail below with reference to FIGS. 6D and 6E.

According to an embodiment, a light emitting element may be disposed in the second region 62 on one side (e.g., surface) of the circuit board 450. For example, the light emitting element (e.g., UV light emitting element) in the second region 62 may emit UV light. UV light emitted from the light emitting element (e.g., UV light emitting element) in the second region 62 may be directed toward a user's body (e.g., skin).

According to an embodiment, UV light emitted from the light emitting element (e.g., UV light emitting element) in the second region 62 may be prevented (e.g., blocked or suppressed) from being transmitted to the light receiving element in the ninth region 69 by the barrier member 460.

According to an embodiment, the rear glass 470 may include a region 73 corresponding to the barrier member 460 disposed on one side (e.g., surface) of the circuit board 450. The rear glass 470 may further include regions corresponding to the second region 62 where the light emitting element is disposed and the ninth region 69 where the light receiving element is disposed, and those regions may be referred to as windows.

FIG. 6B illustrates the A-A′ cross-section passing through the second region 62 and the ninth region 69 in FIG. 6A but, without limitation thereto, may show a cross-section passing through the first region 61 and the fifth region 65, a cross-section passing through the first region 61 and the ninth region 69, and a cross-section passing through the sixth region 66 and the fifth region 65 in FIG. 6A.

FIG. 6C is a cross-sectional view illustrating light received by a light receiving element based on light emitted from a light emitting element according to an embodiment. FIG. 6C may correspond to the A-A′ cross-section of FIG. 6A but, without limitation thereto, may correspond to a cross-section passing through the first region 61 and the fifth region 65, a cross-section passing through the first region 61 and the ninth region 69, and a cross-section passing through the sixth region 66 and the fifth region 65 in FIG. 6A.

In FIG. 6C, light may be emitted from the light emitting element disposed in the second region 62 on one side (e.g., surface) of the circuit board 450. For example, the light emitting element (e.g., UV light emitting element) in the second region 62 may emit UV light toward the glass 470. Direct arrival of the emitted UV light to the light receiving element disposed in the ninth region 69 may be blocked by the barrier member 460, but reflected light of the emitted UV light may still reach the light receiving element by avoiding the barrier member 460. For example, the emitted UV light may be reflected by the interface between the glass 470 and the skin (SKIN), and the reflected light \textcircled{1} may reach the light receiving element disposed in the ninth region 69. The emitted UV light may be reflected by various substances in the skin (SKIN), and the reflected light \textcircled{2} may reach the light receiving element disposed in the ninth region 69. The emitted UV light may react with AGEs in the skin (SKIN) to generate a fluorescence signal \textcircled{3}, and the generated fluorescence signal \textcircled{3} may reach the light receiving element disposed in the ninth region 69.

An electronic device according to an embodiment may analyze the fluorescence signal \textcircled{3} to estimate the amount of AGEs in the skin (SKIN).

FIG. 6D is a cross-sectional view illustrating a light-filtering member of an electronic device according to an embodiment. FIG. 6D may correspond to the cross-sectional view illustrating FIG. 6C, but the disclosure is not limited thereto. For example, FIG. 6D may correspond to the A-A′ cross-section of FIG. 6A, a cross-section passing through the first region 61 and the fifth region 65 in FIG. 6A, a cross-section passing through the first region 61 and the ninth region 69, and a cross-section passing through the sixth region 66 and the fifth region 65.

An electronic device according to an embodiment may include a light-filtering member 481. The light-filtering member 481 may be formed on a light receiving element. For example, the light-filtering member 481 may be formed on the light receiving elements disposed in the ninth region 69 and the fifth region 65 of FIGS. 5 and 6A. According to an embodiment, the light-filtering member 481 may be formed on a plurality of light receiving elements. In case that the circuit board 450 is viewed vertically (e.g., from the top), the light-filtering member 481 formed on the light receiving element may have the shape of the light receiving element. For example, in case that the circuit board 450 is viewed vertically, the light-filtering member 481 may have a rectangular shape.

In an embodiment, the light-filtering member 481 may filter out light having a wavelength shorter than a designated wavelength to prevent (e.g., block or control) that light from entering the light receiving element. For example, the light-filtering member 481 may block light of a specific wavelength band by absorbing light of a specific wavelength band and passing light of another wavelength band but, without limitation thereto, may block light of a specific wavelength band using various principles.

In an embodiment, the light-filtering member 481 functions as a long pass filter and may block light of a wavelength shorter than a cut-on wavelength and transmit light of a wavelength longer than the cut-on wavelength. An ideal long pass filter may block all light of wavelengths shorter than the cut-on wavelength and pass all light of wavelengths longer than the cut-on wavelength. However, an actual filter has about 50% transmittance at the cut-on wavelength and has a changing gradient in the transition region that represents the wavelength range between the blocking section and the transmission section. In other words, the narrower the width of the transition region of the filter, the closer it is to an ideal filter.

In the disclosure, the expression of blocking light of a wavelength shorter than a specific wavelength or the expression of passing light of a wavelength longer than a specific wavelength may be interpreted as the specific wavelength being a cut-on wavelength where about 50% of the light is transmitted at the specific wavelength.

According to an embodiment, the light-filtering member 481 may block light having a wavelength shorter than a wavelength included in a range of about 480 nm to 520 nm, thereby preventing (e.g., blocking or controlling) that light from entering the light receiving element. According to an embodiment, the light-filtering member 481 may block light having a wavelength shorter than about 500 nm.

According to an embodiment, the light-filtering member 481 may be laminated on the light receiving element through thermal evaporation but, without limitation thereto, other coating, deposition, or lamination methods may be used. The light-filtering member 481 may be formed by depositing silicon dioxide (SiO₂) and silicon nitride (SiN) on the light receiving element through thermal evaporation but, without limitation thereto, other materials may be used. The light-filtering member 481 may be a coating layer or film formed on the light receiving element.

According to an embodiment, in case that the circuit board 450 is viewed vertically, the area of the light-filtering member 481 may be substantially the same as the area of the light receiving element on which the light-filtering member 481 is formed.

According to an embodiment, the reflected lights (\textcircled{1} and \textcircled{2} of FIG. 6C) of UV light are blocked by the light-filtering member 481, and their entry into the light receiving element is prevented (e.g., blocked or controlled). Since the reflected lights (\textcircled{1} and \textcircled{2} of FIG. 6C) of UV light having relatively large intensity are difficult to reach the light receiving element, the fluorescence signal \textcircled{3} generated by AGEs having relatively small intensity may be measured more accurately.

FIG. 6E is a cross-sectional view illustrating two light-filtering members of an electronic device according to an embodiment. FIG. 6E may correspond to the cross-sectional views illustrating FIGS. 6B and 6D, but the disclosure is not limited thereto. For example, FIG. 6E may correspond to the A-A′ cross-section of FIG. 6A, a cross-section passing through the first region 61 and the fifth region 65 in FIG. 6A, a cross-section passing through the first region 61 and the ninth region 69, and a cross-section passing through the sixth region 66 and the fifth region 65.

An electronic device according to an embodiment may include two light-filtering members 481, 482. The first light-filtering member 481 may be formed on a light receiving element. For example, the first light-filtering member 481 may be formed on the light receiving elements disposed in the ninth region 69 and the fifth region 65 of FIGS. 5 and 6A. According to an embodiment, the first light-filtering member 481 may be formed on a plurality of light receiving elements. In case that the circuit board 450 is viewed vertically, the first light-filtering member 481 formed on the light receiving element may have the shape of the light receiving element. For example, the first light-filtering member 481 may have a rectangular shape.

In an embodiment, the second light-filtering member 482 may be formed on a region corresponding to the light receiving element in the glass 470. The region corresponding to the light receiving element in the glass 470 may correspond to one of the regions (63, 65, 67, and 69 of FIGS. 5 and 6A) where the light receiving element is disposed on the circuit board 450. In case that the circuit board is viewed vertically, the region corresponding to the light receiving element in the glass 470 may have a rectangular shape. The glass 470 may include a plurality of windows corresponding to the plurality of regions (63, 65, 67, 69 of FIGS. 5 and 6A) where light receiving elements are disposed on one side (e.g., surface) of the circuit board 450. The light receiving element may be disposed in one of the plurality of windows in the glass 470. The second light-filtering member 482 may be formed on one side (e.g., surface) of the window corresponding to the light receiving element in the glass 470.

In an embodiment, the first and second light-filtering members 481, 482 may filter out light having a wavelength shorter than a designated wavelength to prevent (e.g., block or control) that light from entering the light receiving element. For example, the first and second light-filtering members 481, 482 may block light of a specific wavelength band by absorbing light of a specific wavelength band and passing light of another wavelength band, but are not limited thereto, and may block light of a specific wavelength band using various principles.

According to an embodiment, the first and second light-filtering members 481, 482 function as long pass filters and may block light of a wavelength shorter than a cut-on wavelength and transmit light of a wavelength longer than the cut-on wavelength.

According to an embodiment, the first and second light-filtering members 481, 482 may block light having a wavelength shorter than a wavelength included in a range of about 480 nm to 520 nm, thereby preventing (e.g., blocking or controlling) that light from entering the light receiving element. According to an embodiment, the first and second light-filtering members 481, 482 may block light having a wavelength shorter than about 500 nm. According to an embodiment, the cut-on wavelengths of the first and second light-filtering members 481, 482 may be different, thereby enabling UV light to be blocked over a wider range of wavelength bands.

According to an embodiment, the first light-filtering member 481 may be laminated on the light receiving element through thermal evaporation, and the second light-filtering member 482 may be laminated on the glass 470 through sputtering but, without limitation thereto, other coating, deposition, or lamination methods may be used. The first light-filtering member 481 may be formed by depositing silicon dioxide (SiO₂) and silicon nitride (SiN) on the light receiving element through thermal evaporation, and the second light-filtering member 482 may be formed by depositing silicon dioxide (SiO₂) and niobium pentoxide (Nb₂O₅) on the glass 470 through sputtering but, without limitation thereto, other materials may be used. The first and second light-filtering members 481, 482 may be coating layers or films formed on the light receiving element and the glass 470, respectively.

According to an embodiment, the first and second light-filtering members 481, 482 may be spaced apart by a distance D within a range of about 0.36 mm to 0.37 mm. For example, the first and second light-filtering members 481, 482 may be spaced apart by about 0.368 mm from each other. Since the first and second light-filtering members 481, 482 may generate heat by absorbing UV light, the first and second light-filtering members 481, 482 may be spaced apart rather than attached to each other to reduce heat accumulation and maintain the lifespan and performance of the first and second light-filtering members 481, 482.

According to an embodiment, in case that the circuit board 450 is viewed vertically (e.g., top view), the area of the first light-filtering member 481 may be substantially the same as the area of the light receiving element on which the first light-filtering member 481 is formed, and the area of the second light-filtering member 482 may be wider than the area of the light receiving element and the area of the first light-filtering member 481. As a result, not only UV light incident perpendicular to the light receiving element but also UV light incident at an angle may be effectively blocked. According to an embodiment, the second light-filtering member 482 may cover a wider area than the first light-filtering member 481 to block UV light.

According to an embodiment, the reflected lights (\textcircled{1} and \textcircled{2} of FIG. 6C) of UV light are blocked by the first light-filtering member 481 and the second light-filtering member 482, and their entry into the light receiving element is prevented (e.g., blocked or controlled). Since the reflected lights (\textcircled{1} and \textcircled{2} of FIG. 6C) of UV light having relatively large intensity are difficult to reach the light receiving element, the fluorescence signal \textcircled{3} generated by AGEs having relatively small intensity may be measured more accurately.

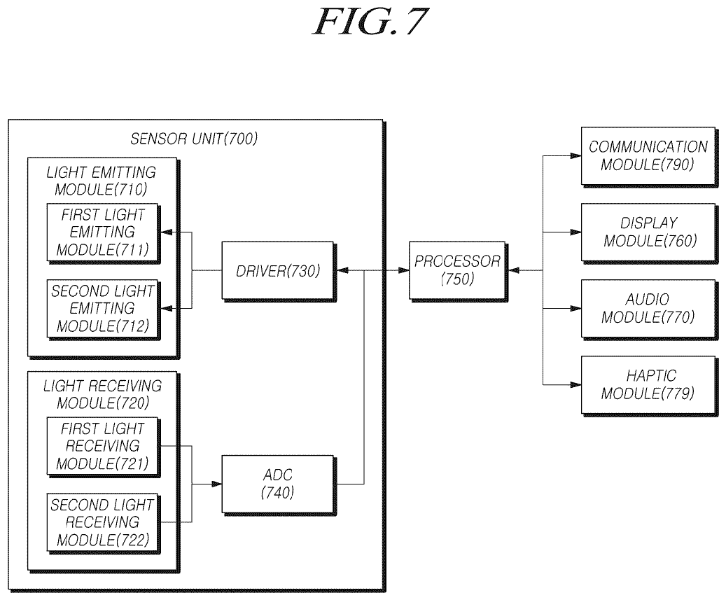

FIG. 7 is a block diagram illustrating an example configuration for biometric information acquisition, according to an embodiment.

In FIG. 7, an electronic device (e.g., the electronic device 101 of FIG. 1) may include at least one of a sensor unit 700 (e.g., the sensor module 176 of FIG. 1), a processor (e.g., including processing circuitry) 750 (e.g., the processor 120 of FIG. 1), a display module (e.g., including a display) 760 (e.g., the display module 160 of FIG. 1), an audio module (e.g., including circuitry) 770 (e.g., the audio module 170 of FIG. 1), a haptic module (e.g., including haptic circuitry) 779 (e.g., the haptic module 179 of FIG. 1), and/or a communication module (e.g., including communication circuitry) 790 (e.g., the communication module 190 of FIG. 1).

In an embodiment, the sensor unit 700 may include at least one of a light emitting module 710, a light receiving module 720, a driver 730, or an analog digital converter (ADC) 740. Some or all of the components included in the sensor unit 700 may be included in the processor 750. The light emitting module 710 may include light emitting elements disposed in some or all of the plurality of regions 61, 62, 63, 64, 65, 66, 67, 68, 69 illustrated in FIG. 6A. The light receiving module 720 may include light receiving elements disposed in some or all of the plurality of regions 61, 62, 63, 64, 65, 66, 67, 68, 69 illustrated in FIG. 6A.

In an embodiment, the light emitting module 710 may emit light. The light emitting module 710 may include a first light emitting module 711 and a second light emitting module 712. The light emitting module 710 may emit light under the control of the driver (e.g., including various circuitry) 730. Each of the first light emitting module 711 and the second light emitting module 712 may include one or more light emitting elements capable of emitting light. The first light emitting module 711 may include light emitting elements disposed in the second region 62 and/or the sixth region 66 illustrated in FIG. 6A. The second light emitting module 712 may include light emitting elements disposed in some or all of the plurality of regions 61, 62, 63, 64, 65, 66, 67, 68, 69. Each of the first light emitting module 711 and the second light emitting module 712 may emit light under the control of the driver 730. Light emitted by the first light emitting module 711 may be used to determine whether the electronic device 101 contacts at least a portion of a user's body. In an embodiment, the first light emitting module 711 may include one or more first light emitting elements. The first light emitting element may include a UV light emitting element. The UV light emitting element may be an LED or laser that emits UV light in a wavelength band of about 320 nm to 365 nm. The peak wavelength of light emitted by the UV light emitting element may be included in a range of about 320 nm to 365 nm, but the disclosure is not limited thereto. For example, the peak wavelength or center wavelength of light emitted by the UV light emitting element may be included in a range of about 360 nm to 370 nm.

In an embodiment, light emitted by the second light emitting module 712 may be used to obtain biometric information of a user. For example, the biometric information may include information such as the amount of biological substances in the user's body (e.g., AGEs value). In an embodiment, the second light emitting module 712 may include one or more UV light emitting elements. Some or all of the light emitting elements included in the first light emitting module 711 may be included in the second light emitting module 712.

In an embodiment, the driver 730, which may include various circuitry, may transmit a signal controlling the light emitting module 710. The driver 730 may supply current and/or voltage for the light emitting module 710 to operate. The driver 730 may supply current and/or voltage so that the first light emitting module 711 and the second light emitting module 712 each operate independently.

In an embodiment, the light receiving module 720 may receive light. The light receiving module 720 may include a first light receiving module 721 and a second light receiving module 722. Each of the first light receiving module 721 and the second light receiving module 722 may include one or more light receiving elements capable of receiving light (or optical signals). The first light receiving module 721 may include light receiving elements disposed in the third region 63, the fifth region 65, the seventh region 67, and/or the ninth region 69 illustrated in FIG. 6A. The second light receiving module 722 may include light receiving elements disposed in the third region 63, the fifth region 65, the seventh region 67, and/or the ninth region 69 illustrated in FIG. 6A. The first light receiving module 721 may receive light in response to light emission by the first light emitting module 711. The first light receiving module 721 may receive light while the first light emitting module 711 emits light. Light received by the first light receiving module 721 may be used to determine whether the electronic device 101 contacts at least a portion (e.g., part) of a user's body. The first light receiving module 721 may include a first light receiving element and a second light receiving element. The first light receiving element may be a light receiving element (e.g., UV cut PD) with a light-filtering member formed thereon. The second light receiving element may be a light receiving element (e.g., Normal PD) without a light-filtering member formed thereon. Since the first light receiving element receives light filtered by the light-filtering member and the second light receiving element receives light not filtered by the light-filtering member, the wavelength band of light received by the first light receiving element may be narrower than the wavelength band of light received by the second light receiving element. For example, the wavelength band of light received by the first light receiving element may be within a range of about 480 nm to 980 nm, and the wavelength band of light received by the second light receiving element may be within a range of about 340 nm to 980 nm. However, the disclosure is not limited thereto. The first light receiving element may receive first light in response to light emission by the first light emitting module 711. The first light may include first light (e.g., fluorescence signal) generated as a result of an interaction between light emitted by the first light emitting module 711 and a body of a user (e.g., a user wearing a wearable device). The second light receiving element may receive second light in response to light emission by the first light emitting module 711. The second light may include reflected light where light emitted by the first light emitting module 711 is reflected from the body.

In an embodiment, the second light receiving module 722 may receive light in response to light emission by the second light emitting module 712. The second light receiving module 722 may receive light while the second light emitting module 712 emits light. Light received by the second light receiving module 722 may be used to obtain biometric information. Some or all of the light emitting elements included in the first light receiving module 721 may be included in the second light receiving module 722. The second light receiving module 722 may include a light receiving element with a light-filtering member formed thereon.

In an embodiment, the light receiving module 720 may transmit (e.g., analog voltage or current) corresponding to the received light to the ADC 740. Each of the first light receiving module 721 and the second light receiving module 722 may transmit analog voltage or current corresponding to the received first light and second light to the ADC 740.

In an embodiment, the ADC 740 may include various circuitry and convert an analog signal (e.g., analog voltage or current) received from the light receiving module 720 into a digital signal (or digital value). The ADC 740 may convert analog signals received from each of the first light receiving module 721 and the second light receiving module 722 into digital signals. The ADC 740 may transmit the converted signal to the processor 750.

In an embodiment, the processor 750 may include various processing circuitry (e.g., the descriptions of the processor 120 above apply equally to the processor 750 and may not be repeated here) and control the driver 730. The processor 750 may control the light emitting module 710 through the driver 730. The processor 750 may control the first light emitting module 711 to emit light. The processor 750 may control the second light emitting module 712 to emit light. The processor 750 may obtain a value corresponding to light received by the light receiving module 720 through the ADC 740. The processor 750 may determine whether the electronic device 101 contacts at least a portion of a user's body using light received by the light receiving module 720. The processor 750 may determine whether the electronic device 101 contacts at least a portion (e.g., part) of a user's body using light received by the first light receiving module 721 while the first light emitting module 711 emits light. The processor 750 may receive light using the second light receiving module 722 based on determining that the electronic device 101 contacts at least a portion of a user's body. The processor 750 may obtain biometric information using light received by the second light receiving module 722 while the second light emitting module 712 emits light. The processor 750 may provide feedback based on determining that the electronic device 101 does not contact at least a portion of a user's body.

In an embodiment, the processor 750 may control at least one of the display module 760, the audio module 770, the haptic module 779, or the communication module 790. The processor 750 may control at least one of the display module 760, the audio module 770, the haptic module 779, or the communication module 790 using light received by the first light receiving module 721 and/or the second light receiving module 722. The processor 750 may provide feedback through at least one of the display module 760, the audio module 770, the haptic module 779, or the communication module 790 based on determining that the electronic device 101 does not contact at least a portion of a user's body. The electronic device 101 may transmit information about light received by the first light receiving module 721 and/or the second light receiving module 722 to an external electronic device (e.g., the external electronic devices 102 and/or 104 of FIG. 1) and/or a server (e.g., the server 108 of FIG. 1) connected to the electronic device 101 through the communication module 790.

In an embodiment, the display module 760, the audio module 770, the haptic module 779, and the communication module 790 may respectively be the display module 160, the audio module 170, the haptic module 179, and the communication module 190 of FIG. 1. Redundant descriptions of the display module, audio module, haptic module, and communication module are omitted.

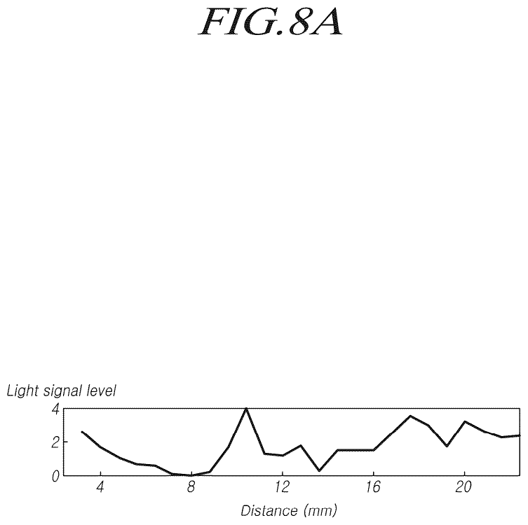

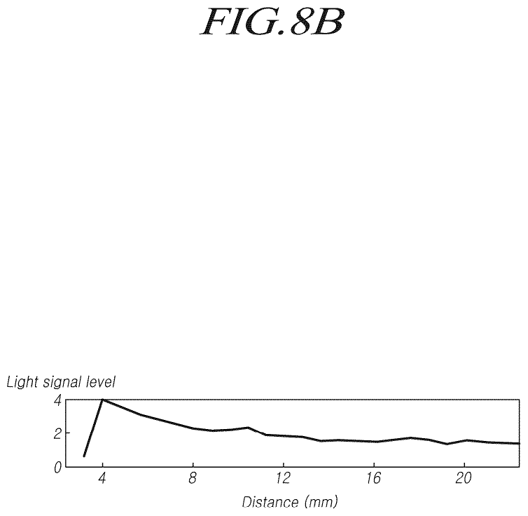

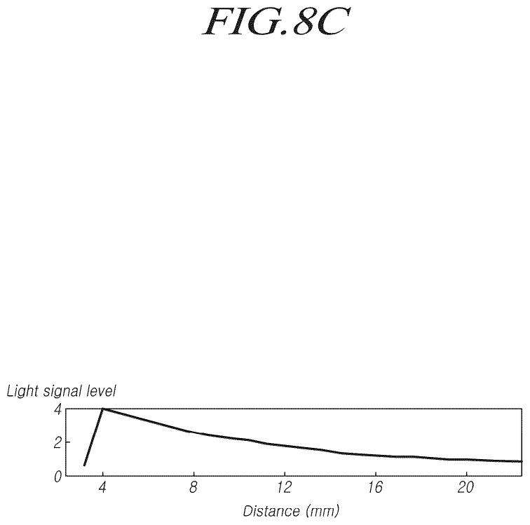

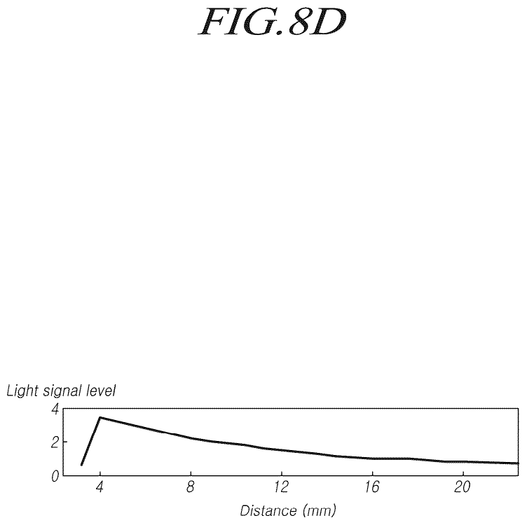

FIGS. 8A, 8B, 8C and 8D are graphs describing light received by a light receiving element, according to an embodiment.

In FIGS. 8A to 8D, the electronic device 101 may include a sensor for measuring light. The electronic device 101 may measure light intensity while gradually increasing the distance between the electronic device 101 and a user. The electronic device 101 may be fixed in a predetermined position using a jig. The electronic device 101 may measure light intensity while increasing the gap with the user from the fixed position. For example, the sensor may include a light emitting module (e.g., the first light emitting module 711 of FIG. 7), a light receiving module (e.g., the first light receiving module 721 of FIG. 7), and an analog digital converter (ADC) (e.g., the ADC 740 of FIG. 7). The light emitting module may include a first light emitting element, a second light emitting element, and a third light emitting element. The first light emitting element may emit light having a peak wavelength of about 365 nm. The second light emitting element may be an IR light emitting element that emits light having a wavelength within a range of about 700 nm or more. The third light emitting element may be a red light emitting element that emits light having a wavelength within a range of about 620 nm to 720 nm. The light receiving module may include a first light receiving element and a second light receiving element. The first light receiving element may receive light of a first wavelength band including a range of 480 nm to 980 nm. A light-filtering member that blocks light having a wavelength shorter than a wavelength included in a range of about 480 nm to 520 nm may be formed on the first light receiving element. The second light receiving element may receive light of a second wavelength band including a range of 340 nm to 980 nm. A light-filtering member may not be formed on the second light receiving element.

As illustrated in FIG. 6C, light emitted by the light emitting element may be reflected by a user's body. Reflected light (R) reflected by the user's body may include R_{direct} (e.g., reflected light \textcircled{1} of FIG. 6C), which is a skin surface reflection component, and R_{indirect} (e.g., reflected light \textcircled{2} of FIG. 6C), which is an in-skin reflection component. In a contact state, R_{direct} is relatively small and, in a state in which the sensor is close to the skin surface, R_{direct} is relatively large. The intensity of reflected light (R) may be relatively small in a contact state, increase up to a predetermined distance, and then decrease as the distance between the sensor and the skin surface increases. The electronic device 101 may determine whether the electronic device 101 is close to a user's body using reflected light (R). Light emitted by the light emitting element may react with a user's body (e.g., fluorescent substances in the body) to generate a fluorescence signal (e.g., the fluorescence signal \textcircled{3} of FIG. 6C). The fluorescence signal \textcircled{3} has smaller light intensity compared to the reflected lights \textcircled{1} and \textcircled{2}.

In an embodiment, the electronic device 101 may measure four types of light including first light, second light, third light, and fourth light using the sensor. The first light is light received by the first light receiving element in response to light emission by the first light emitting element. Reflected light of light emitted by the first light emitting element may be blocked by the light-filtering member and prevented (e.g., blocked or controlled) from entering the first light receiving element. The first light receiving element may substantially receive a fluorescence signal (e.g., a fluorescence signal generated by AGEs). The first light may substantially include a fluorescence signal. The second light is light received by the second light receiving element in response to light emission by the first light emitting element. The third light is light received by the second light receiving element in response to light emission by the second light emitting element. The fourth light is light received by the second light receiving element in response to light emission by the third light emitting element. The second light receiving element may receive reflected light and a fluorescence signal. The second light, third light, and fourth light may include reflected light and a fluorescence signal. Light received by the light receiving element may be converted to an optical signal level by the ADC. The first light, second light, third light, and fourth light may be converted by the ADC into a first optical signal level, a second optical signal level, a third optical signal level, and a fourth optical signal level, respectively. The intensity of the first light, the intensity of the second light, the intensity of the third light, and the intensity of the fourth light may correspond to the first optical signal level, the second optical signal level, the third optical signal level, and the fourth optical signal level, respectively.

In the graphs of FIGS. 8A to 8D, the horizontal axis represents the distance (measured and/or represented in mm) between the sensor and the user, and the vertical axis represents the measured optical signal level. The graph of FIG. 8A is for the first light, with the horizontal axis representing the distance between the sensor and the user, and the vertical axis representing the first optical signal level. The graph of FIG. 8B is for the second light, with the horizontal axis representing the distance between the sensor and the user, and the vertical axis representing the second optical signal level. The graph of FIG. 8C is for the third light, with the horizontal axis representing the distance between the sensor and the user, and the vertical axis representing the third optical signal level. The graph of FIG. 8D is for the fourth light, with the horizontal axis representing the distance between the sensor and the user, and the vertical axis representing the fourth optical signal level.

In FIG. 8A, the first optical signal level decreases in intensity as the distance increases up to about 8 mm, then increases in intensity up to about 10 mm, and thereafter illustrates a tendency for the intensity to vary independently of distance.

In FIGS. 8B to 8D, the second optical signal level, the third optical signal level, and the fourth optical signal level form a peak in case that the distance is a first distance, and the intensity decreases thereafter. The first distance may be about 4 mm, but the disclosure is not limited thereto. The electronic device 101 may determine whether the electronic device 101 is close to a user's body based on the first distance. In case that the distance between the electronic device 101 and the user's body is equal to or less than the first distance, the electronic device 101 may determine that the electronic device 101 is in a state of being close to the user's body. In case that the distance between the electronic device 101 and the user's body exceeds the first distance, the electronic device 101 may determine that the electronic device 101 is not in a state of being close to the user's body.

In an embodiment, the graph for the first light substantially including a fluorescence signal (e.g., FIG. 8A) may show a different aspect from the graphs for the second light, third light, and fourth light including reflected light having greater light intensity than the fluorescence signal as well as the fluorescence signal (e.g., FIGS. 8B, 8C, and 8D).

In an embodiment, the electronic device 101 may determine whether the electronic device 101 is close to a user's body using reflected light (e.g., second light, third light, and fourth light), but it may be relatively difficult to determine whether the electronic device 101 contacts the user's body.

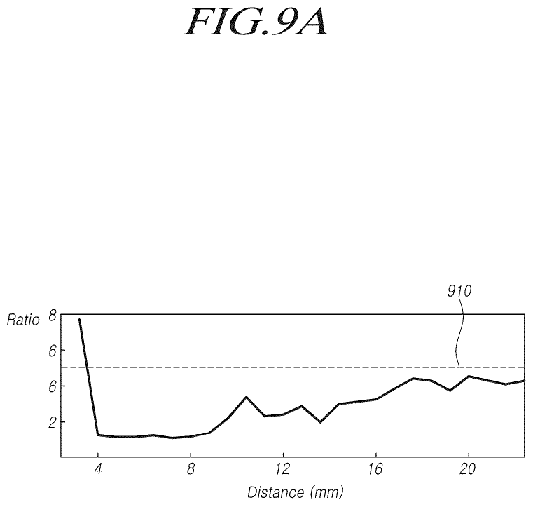

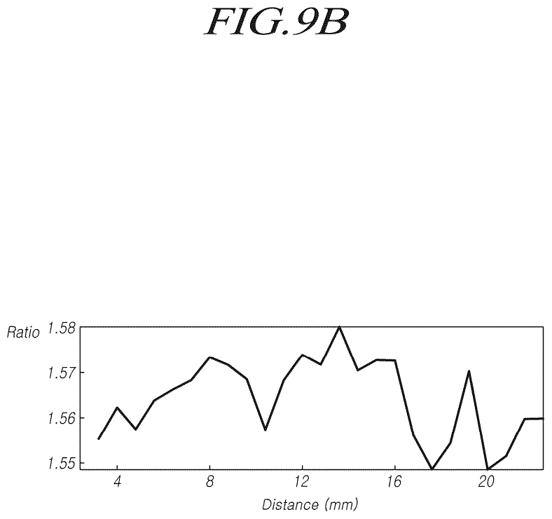

FIGS. 9A and 9B are graphs describing an example method of determining a contact state using a pair of optical signals, according to an embodiment.

In the graphs of FIGS. 9A and 9B, the horizontal axis represents the distance (measured and/or represented in mm) between the sensor and the user's body, and the vertical axis represents the ratio of measured optical signal levels. The graph of FIG. 9A illustrates on the vertical axis a first ratio obtained by dividing the first optical signal level of FIG. 8A by the second optical signal level of FIG. 8B along the horizontal axis of distance. The graph of FIG. 9B illustrates on the vertical axis a second ratio obtained by dividing the third optical signal level of FIG. 8C by the fourth optical signal level of FIG. 8D along the horizontal axis of distance. The first ratio may correspond to a ratio obtained by dividing the intensity of the first light by the intensity of the second light. The second ratio may correspond to a ratio obtained by dividing the intensity of the third light by the intensity of the fourth light.

In FIG. 9A, the electronic device 101 may determine whether the electronic device 101 contacts at least a portion (e.g., part) of a user's body based on the first light and the second light. The electronic device 101 may determine whether the electronic device 101 contacts at least a portion of a user's body based on the first ratio. The electronic device 101 may determine that the electronic device 101 contacts at least a portion of a user's body in case that the distance between the electronic device 101 and at least a portion of the user's body is equal to or less than a second distance. The first ratio may have a value equal to or greater than a threshold 910 in case that the distance is equal to or less than the second distance. The first ratio may have a value less than the threshold 910 in case that the distance exceeds the second distance. The electronic device 101 may determine that the electronic device 101 contacts at least a portion of a user's body in case that the first ratio is equal to or greater than the threshold 910. The electronic device 101 may determine that the electronic device 101 does not contact at least a portion of a user's body in case that the first ratio is less than the threshold 910.

In FIG. 9B, it may be relatively difficult to determine whether the electronic device 101 contacts at least a portion of a user's body using the third light and the fourth light. The third light and the fourth light are light emitted by different light emitting elements and received by the same type of light receiving element. On the other hand, the first light and the second light are light emitted by the same type of light emitting element and received by different light receiving elements.

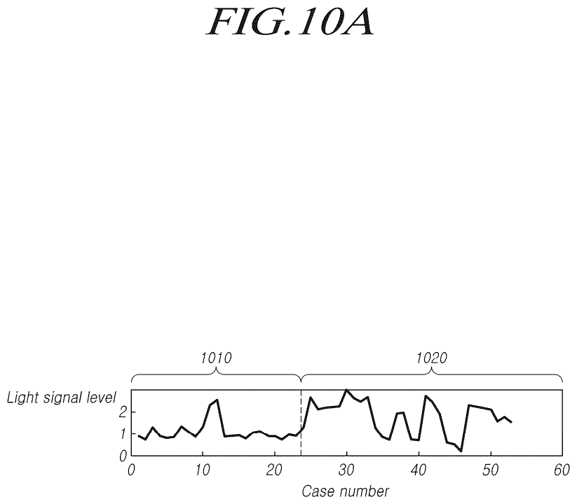

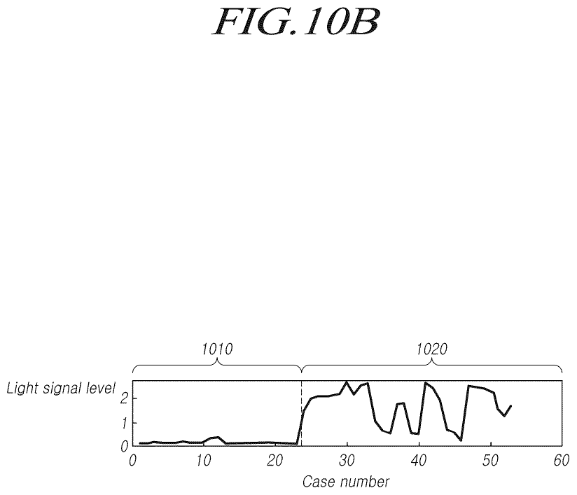

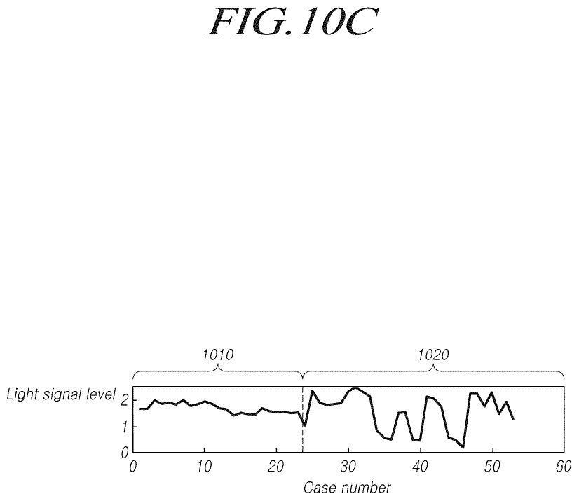

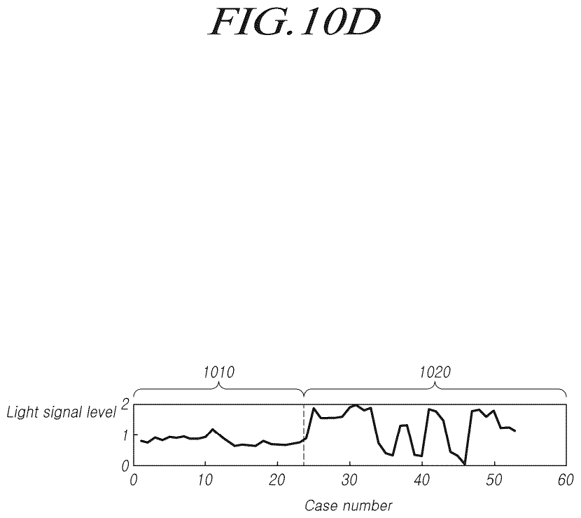

FIGS. 10A, 10B, 10C and 10D are graphs describing light obtained by a light receiving element, according to an embodiment.

The graphs of FIGS. 10A to 10D show examples of the first optical signal level, the second optical signal level, the third optical signal level, and the fourth optical signal level measured by the sensor in case that the distance between the surface of the sensor and the body surface is about 0 mm (contact state) and in case that it exceeds 0 mm (non-contact state). For descriptions of the first optical signal level, the second optical signal level, the third optical signal level, and the fourth optical signal level, refer to FIGS. 8A to 8D.

In the graphs of FIGS. 10A to 10D, the horizontal axis represents the case number, and the vertical axis represents the measured optical signal level. Contact state cases are displayed in the first section 1010, and non-contact state cases are displayed in the second section 1020. There may be about 20 or more contact state cases. There may be about 20 or more non-contact state cases. The graph of FIG. 10A illustrates the first optical signal level for contact state cases and non-contact state cases. The graph of FIG. 10B illustrates the second optical signal level for contact state cases and non-contact state cases. The graph of FIG. 10C illustrates the third optical signal level for contact state cases and non-contact state cases. The graph of FIG. 10D illustrates the fourth optical signal level for contact state cases and non-contact state cases. The distance between the surface of the sensor and the body surface may be different in each non-contact state case.

In FIG. 10A, the first optical signal level value detected in the contact state case may be detected in the non-contact state case. In FIG. 10B, the second signal level value detected in the contact state case may be detected in the non-contact state case. In FIG. 10C, the third optical signal level value detected in the contact state case may be detected in the non-contact state case. In FIG. 10D, the fourth optical signal level value detected in the contact state case may be detected in the non-contact state case. It is difficult to distinguish between contact state cases and non-contact state cases using the intensity of each of the first light, second light, third light, and fourth light.

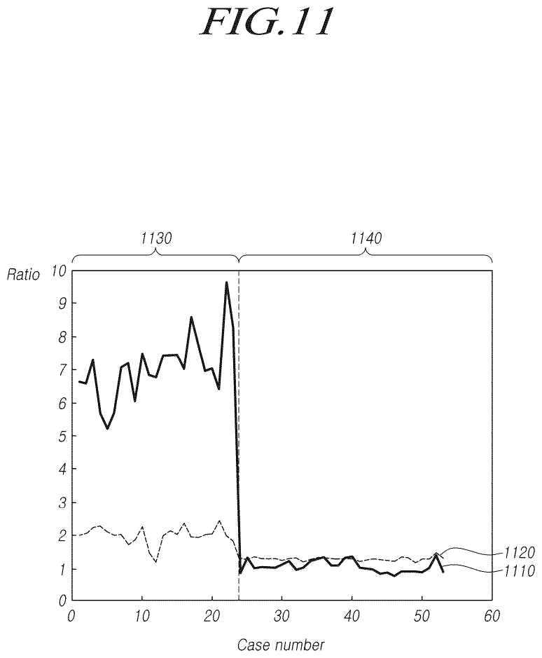

FIG. 11 is a graph describing an example method of determining a contact state using a pair of optical signals, according to an embodiment.

In the graph of FIG. 11, the horizontal axis represents the case number, and the vertical axis represents the ratio of measured optical signal levels. In the graph of FIG. 11, graph 1110 illustrates on the vertical axis a first ratio obtained by dividing the first optical signal level of FIG. 10A by the second optical signal level of FIG. 10B along the horizontal axis of case number. Graph 1120 illustrates on the vertical axis a second ratio obtained by dividing the third optical signal level of FIG. 10C by the fourth optical signal level of FIG. 10D along the horizontal axis of case number. Contact state cases are displayed in the first section 1130, and non-contact state cases are displayed in the second section 1140.

According to graph 1110, the electronic device 101 may determine whether the electronic device 101 contacts at least a portion (e.g., part) of a user's body based on the first light and the second light. The electronic device 101 may more clearly distinguish between contact and non-contact states using the first ratio. The electronic device 101 may distinguish between contact and non-contact states according to whether the first ratio is equal to or greater than a threshold. The electronic device 101 may identify a contact state in case that a first ratio equal to or greater than the threshold is obtained. The electronic device 101 may identify a non-contact state in case that a first ratio less than the threshold is obtained. For example, a value between the minimum y-value (e.g., about 5) of graph 1110 in the first section 1130 representing contact state cases and the maximum y-value (e.g., about 1.5) of graph 1110 in the second section 1140 representing non-contact state cases may be set as the threshold. Using the second ratio, it may be relatively difficult to distinguish between contact and non-contact states. According to graph 1120, the second ratio in the first section 1130 representing contact state cases and the second ratio in the second section 1140 representing non-contact state cases are not relatively clearly distinguished based on a specific threshold.

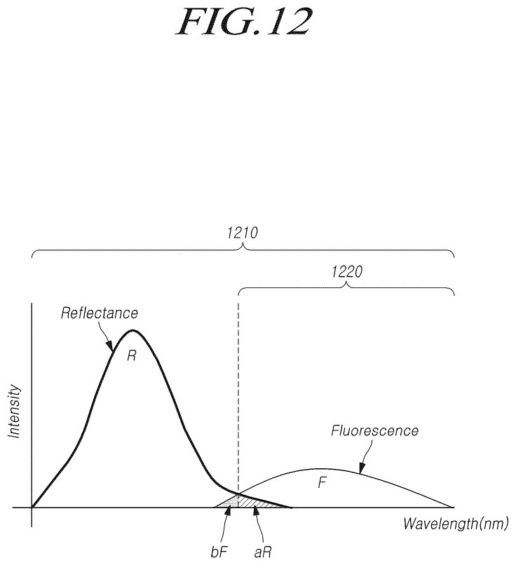

FIG. 12 is a graph describing light received by a light receiving element, according to an embodiment.

In an embodiment, the electronic device 101 may include a first light emitting element, a first light receiving element, and a second light receiving element. For descriptions of the first light emitting element, the first light receiving element, and the second light receiving element, refer to FIGS. 8A to 8D.

In the graph of FIG. 12, the horizontal axis represents the wavelength (nm) of light obtained by the light receiving element, and the vertical axis represents the intensity (lx) of light. The first light emitting element may emit light. Light obtained by each of the first light receiving element and the second light receiving element may be light emitted from a user's body after light emitted by the first light emitting element reaches the user's body. Light emitted by the first light emitting element may be reflected by skin to generate reflected light (R). Light emitted by the first light emitting element may react with skin to generate a fluorescence signal (F). Light emitted from the body may include reflected light (R) and/or a fluorescence signal (F). The Reflectance graph illustrates on the vertical axis the intensity of reflected light (R) measured along the horizontal axis of wavelength. The Fluorescence graph illustrates on the vertical axis the intensity of fluorescence signal (F) measured along the horizontal axis of wavelength.

In an embodiment, the first light obtained by the first light receiving element may include a fluorescence signal (F). The first light receiving element may be configured to receive a fluorescence signal (F) that belongs to a first wavelength band 1220. The first light receiving element may include a light-filtering member on the first light receiving element to receive a fluorescence signal (F). However, the first light receiving element may not receive a portion of the fluorescence signal (F) (e.g., fluorescence signal having a wavelength shorter than the cut-on wavelength) and may receive a portion of the reflected light (R) (e.g., reflected light having a wavelength longer than the cut-on wavelength).

In an embodiment, the second light obtained by the second light receiving element may include reflected light (R) and a fluorescence signal (F). The second light receiving element may be configured to receive a light signal that belongs to a second wavelength band 1210. The second wavelength band 1210 may be wider than the first wavelength band 1220.

In an embodiment, the first light (A) and the second light (B) received by the first light receiving element and the second light receiving element may be represented by Equations 1 and 2 below.

$$A = a R + F - bF \qquad [ Equation 1 ]$$

$$B = R + F \qquad [ Equation 2 ]$$

In Equations 1 and 2 above,
A is the first light;
B is the second light;
R is reflected light;
F is the fluorescence signal;
a and b may refer to coefficients.

Equations 1 and 2 above are merely examples to aid understanding, and embodiments of the disclosure may not be limited thereto. For example, Equations 1 and 2 above may be modified, applied, or extended in various ways.

In an embodiment, the first light receiving element may not receive a portion of the fluorescence signal (F) and may receive a portion of the reflected light (R). In this case, each of a and b may be 0 or greater.

In an embodiment, light emitted from a comparative reflector without fluorescent substances after light emitted by the first light emitting element reaches the comparative reflector may be obtained by each of the first light receiving element and the second light receiving element. The first light (A) and the second light (B) may be measured. Light emitted from the comparative reflector does not include a fluorescence signal (e.g., F=0). According to Equations 1 and 2, A=aR and B=R may be obtained. In this case, a=A/B. The coefficient a may be obtained using the first light (A) and the second light (B) emitted from the comparative reflector.

In an embodiment, the coefficient b represents the ratio of the fluorescence signal (bF) not received by the first light receiving element among the fluorescence signal (F). The coefficient b may be optimized using, e.g., the light-filtering member of the first light receiving element. In case that the first light receiving element is configured to receive the fluorescence signal (F) over the entire wavelength band of the fluorescence signal (F), b may be substantially 0.

In an embodiment, the ratio of the first light (A) to the second light (B) may be represented by Equation 3 below.

$$A / B = ( a R + ( 1 - b ) F ) / ( R + F ) \qquad [ Equation 3 ]$$

Equation 3 above is merely an example to aid understanding, and embodiments of the disclosure may not be limited thereto. For example, Equation 3 above may be modified, applied, or extended in various ways.

In an embodiment, in a non-contact state, the reflected light (R) may be relatively large compared to the fluorescence signal (F). In Equation 3, A/B≈aR/R=a. In a skin non-contact state, A/B may be approximated as a.

In an embodiment, in a contact state, the reflected light (R) may be relatively small compared to the non-contact state due to the absence of direct reflected light from the skin surface. In case that the reflected light (R) becomes smaller, the amount of light incident on the skin may increase. In this case, the fluorescence signal (F) emitted by reacting with the skin may become larger. In this case, the reflected light (R) is small compared to the fluorescence signal (F). In Equation 3, A/B≈(1−b)F/F=1−b. In a contact state, A/B may be approximated as 1−b.

In an embodiment, the ratio of the first light (A) to the second light (B) may satisfy the condition of Equation 4 below.

$$0 < a < A / B < 1 - b \qquad [ Equation 4 ]$$

Equation 4 above is merely an example to aid understanding, and embodiments of the disclosure may not be limited thereto. For example, Equation 4 above may be modified, applied, or extended in various ways.

According to Equation 4, the ratio A/B of the first light (A) to the second light (B) may be a value between a and 1−b. In a non-contact state, A/B may be a value similar to a. In a contact state, A/B may be a value similar to 1−b.

As illustrated in FIG. 11, the electronic device 101 may distinguish between contact and non-contact states according to whether the first ratio is equal to or greater than a threshold. The first ratio of FIG. 11 may be substantially A/B. The electronic device 101 may identify a contact state in case that A/B equal to or greater than the threshold is obtained. The electronic device 101 may identify a non-contact state in case that A/B less than the threshold is obtained. The threshold may be any value between a and 1−b.

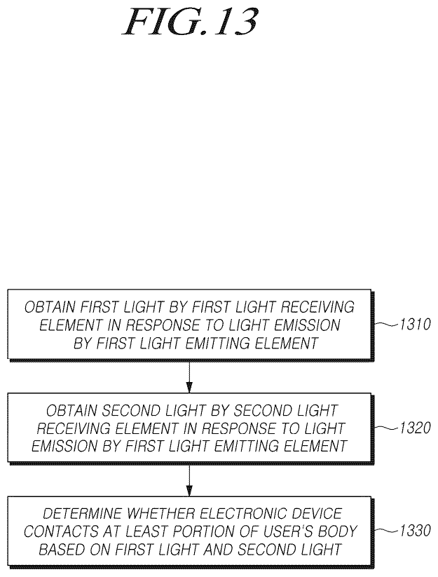

FIG. 13 is a flowchart illustrating an example operation of an electronic device (e.g., the electronic device 101 of FIG. 1) according to an embodiment.

In FIG. 13, the electronic device 101 may include a first light emitting element emitting light, a first light receiving element receiving light of a first wavelength band, and a second light receiving element receiving light of a second wavelength band. The first light emitting element may be a UV light emitting element. The UV light emitting element may be an LED or laser that emits UV light in a wavelength band of about 320 nm to 365 nm. The peak wavelength of light emitted by the UV light emitting element may be included in a range of about 320 nm to 365 nm, but the disclosure is not limited thereto. For example, the peak wavelength or center wavelength of light emitted by the UV light emitting element may be included in a range of about 360 nm to 370 nm. The first light receiving element may be a light receiving element (e.g., UV cut PD) with a light-filtering member formed thereon. The second light receiving element may be a light receiving element (e.g., Normal PD) without a light-filtering member formed thereon. Since the first light receiving element receives light filtered by the light-filtering member and the second light receiving element receives light not filtered by the light-filtering member, the wavelength band of light received by the first light receiving element may be narrower than the wavelength band of light received by the second light receiving element. For example, the wavelength band of light received by the first light receiving element may be within a range of about 480 nm to 980 nm, and the wavelength band of light received by the second light receiving element may be within a range of about 340 nm to 980 nm.

In the following example, each operation may be performed sequentially but is not necessarily performed sequentially. For example, the order of each operation may be changed, or at least two operations may be performed in parallel.

According to an embodiment, operations 1310 to 1330 may be understood as being performed by the processor (e.g., the processor 750 of FIG. 7) of the electronic device 101.

In an embodiment, the electronic device 101 may, in operation 1310, receive first light by the first light receiving element in response to light emission by the first light emitting element. The electronic device 101 may receive first light by the first light receiving element while the first light emitting element emits light. The first light may include at least a portion of light generated by light emitted by the first light emitting element reacting with a user's body.

In an embodiment, the electronic device 101 may, in operation 1320, receive second light by the second light receiving element in response to light emission by the first light emitting element. The electronic device 101 may receive second light by the second light receiving element while the first light emitting element emits light. The second light may include light emitted from the body of the user after light emitted by the first light emitting element reaches the body of the user. The second light may include at least a portion of light generated as a result of an interaction between light emitted by the first light emitting element and the body of the user. The second light may include at least a portion of light reflected from the body of the user by light emitted by the first light emitting element.

In an embodiment, the electronic device 101 may, in operation 1330, determine whether the electronic device contacts at least a portion of a user's body based on the first light and the second light. The electronic device 101 may determine that the electronic device contacts at least a portion of a user's body based on a ratio of the intensity of the first light to the intensity of the second light being equal to or greater than a threshold. The electronic device 101 may determine that the electronic device does not contact at least a portion of a user's body based on a ratio of the intensity of the first light to the intensity of the second light being less than a threshold.

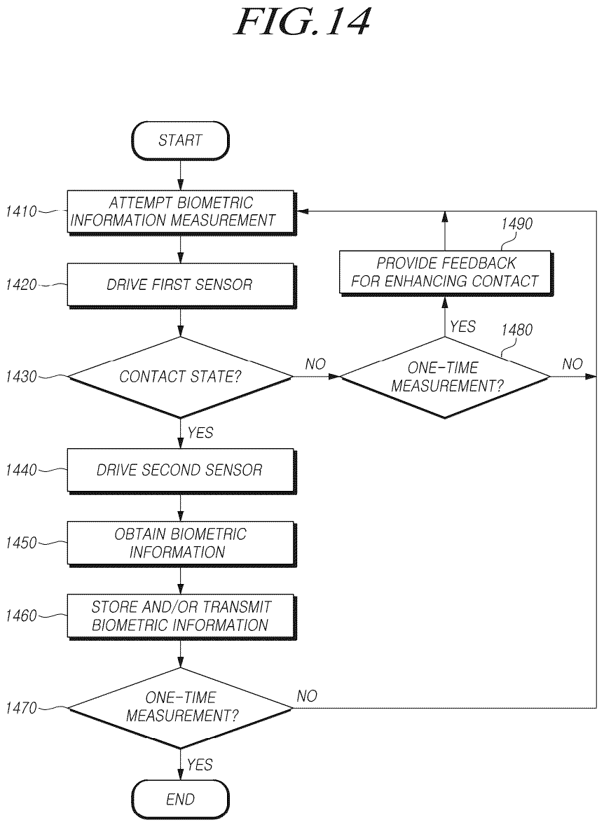

FIG. 14 is a flowchart illustrating an example operation of an electronic device (e.g., the electronic device 101 of FIG. 1) according to an embodiment.

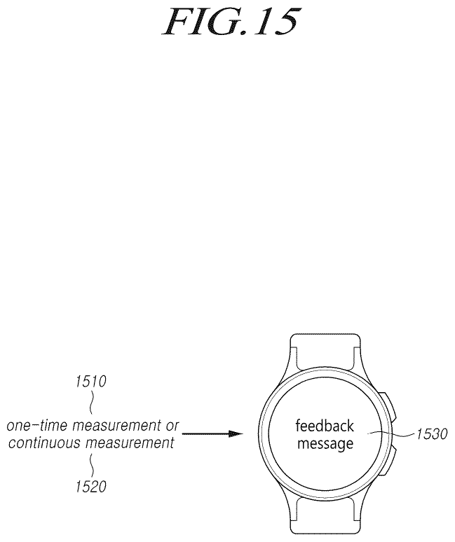

In FIG. 14, the electronic device 101 may obtain biometric information. The electronic device 101 may obtain biometric information by a second sensor (e.g., biometric sensor). The electronic device 101 may drive a first sensor (e.g., contact sensor) to determine whether it is in a contact state before driving the second sensor (e.g., biometric sensor). For example, the first sensor may include the first light emitting module 711 and/or the first light receiving module 721 of FIG. 7. For example, the second sensor may include the second light emitting module 712 and/or the second light receiving module 722 of FIG. 7. A contact state may refer, for example, to a state in which the first sensor included in the electronic device 101 and at least a portion (e.g., part) of a user's body are in contact. A non-contact state may refer, for example, to a state in which the first sensor included in the electronic device 101 and at least a portion of a user's body are not in contact. In an embodiment, the electronic device 101 may drive the second sensor to obtain biometric information based on identifying a contact state. The electronic device 101 may perform different operations according to a measurement mode based on identifying a non-contact state. The electronic device 101 may support a one-time (e.g., single) measurement mode for measuring biometric information once or a continuous measurement mode for continuously measuring biometric information. The electronic device 101 may perform one-time measurement or continuous measurement according to the measurement mode. One-time measurement and continuous measurement is described in greater detail below with reference to FIG. 15. In case that a non-contact state is identified in the electronic device 101 in one-time measurement mode, the electronic device 101 may provide feedback for improving contact. The electronic device 101 may retry biometric information measurement after providing feedback. In case that a contact state is identified in the electronic device 101 in continuous measurement mode, the electronic device 101 may retry biometric information measurement without immediately providing feedback for improving contact.

In the following example, each operation may be performed sequentially but is not necessarily performed sequentially. For example, the order of each operation may be changed, or at least two operations may be performed in parallel.

According to an embodiment, operations 1410 to 1490 may be understood as being performed by the processor (e.g., the processor 750 of FIG. 7) of the electronic device 101.

In an embodiment, the electronic device 101 may, in operation 1410, attempt biometric information measurement. The biometric information may include information such as the amount of biological substances.

In an embodiment, the electronic device 101 may, in operation 1420, drive the first sensor. The electronic device 101 may drive the first sensor in case of attempting biometric information measurement.

In an embodiment, the electronic device 101 may, in operation 1430, determine whether it is in a contact state. The electronic device 101 may determine whether it is in a contact state using the first sensor. The electronic device 101 may determine whether it is in a contact state based on light received by the first light receiving module 721 in response to light emission by the first light emitting module 711. The electronic device 101 may determine whether it is in a contact state based on first light and second light received by the first light receiving element and the second light receiving element included in the first light receiving module 721.

In an embodiment, in case of being in a contact state, the electronic device 101 may, in operation 1440, drive the second sensor.

In an embodiment, the electronic device 101 may, in operation 1450, obtain biometric information. The electronic device 101 may obtain biometric information using the second sensor. The electronic device 101 may obtain biometric information based on light received by the second light receiving module 722 in response to light emission by the second light emitting module 712. For example, the electronic device 101 may obtain an AGE value.

In an embodiment, the electronic device 101 may, in operation 1460, store and/or transmit the biometric information. For example, the electronic device 101 may store the AGE value or transmit it to an external electronic device (e.g., the external electronic devices 102 and/or 104 of FIG. 1) and/or a server (e.g., the server 108 of FIG. 1) connected to the electronic device 101.

In an embodiment, the electronic device 101 may, in operation 1470, determine whether it is a one-time (e.g., single) measurement. In the case of a one-time measurement, the electronic device 101 may terminate the biometric information measurement.

In an embodiment, in case of being not a one-time measurement (if continuous measurement), the electronic device 101 may return to operation 1410.

In an embodiment, in case of being in a non-contact state, the electronic device 101 may, in operation 1480, determine whether it is a one-time measurement.

In an embodiment, in case of being a one-time measurement, the electronic device 101 may, in operation 1490, provide feedback for improving contact. The feedback is described in greater detail below with reference to FIG. 15. In an embodiment, in case of being not a one-time measurement (if continuous measurement), the electronic device 101 may return to operation 1410.

FIG. 15 is a diagram illustrating example feedback provided by an electronic device (e.g., the electronic device 101 of FIG. 1) according to an embodiment.

In FIG. 15, the electronic device 101 may perform one-time measurement 1510 and/or continuous measurement 1520. The electronic device 101 may measure biometric information once through one-time measurement 1510. For example, the electronic device 101 may provide a user interface through which a user may select biometric information (e.g., AGE value) measurement. In case that the user selects biometric information measurement, the electronic device 101 may measure biometric information once.

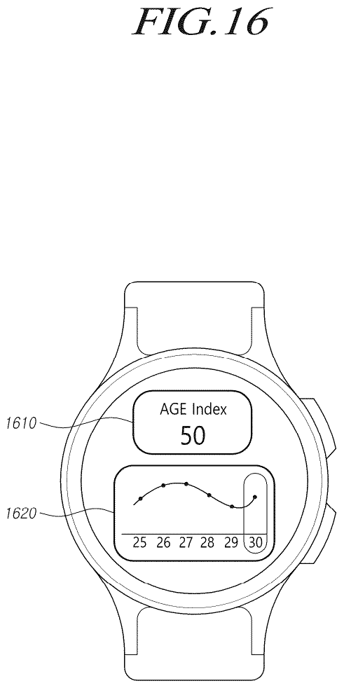

In an embodiment, in case of being a one-time measurement 1510, the electronic device 101 may determine whether the electronic device 101 and at least a portion of a user's body are in contact using the first sensor (e.g., including the first light receiving module 721 of FIG. 7). The electronic device 101 may identify a contact state in which the electronic device 101 and at least a portion of a user's body are in contact, or a non-contact state in which the electronic device 101 and at least a portion of a user's body are not in contact. The electronic device 101 may drive the second sensor (e.g., including the second light receiving module 722 of FIG. 7) to obtain biometric information based on identifying a contact state. The electronic device 101 may provide feedback regarding contact based on identifying a non-contact state. The non-contact state may include a state in which the electronic device (101) is not in functional or sufficient contact with at least a portion of the user's body. For example, the non-contact state may include a case in which the electronic device (101) is completely separated from the user's body, as well as a case in which the degree of contact is below a threshold for providing feedback. The feedback may include a feedback message 1530. For example, the feedback message 1530 may include a message such as “The device is not in contact with skin. Please contact the device with skin and measure again.” According to the feedback message 1530, the user may contact the device with skin and then retry biometric information measurement. The electronic device 101 may determine whether the electronic device 101 and at least a portion (e.g., part) of a user's body are in contact using the first sensor. The electronic device 101 may drive the second sensor to obtain biometric information based on identifying a contact state. The electronic device 101 may provide the obtained biometric information (e.g., see FIG. 16). The electronic device 101 may provide the feedback message 1530 again based on identifying a non-contact state.

In an embodiment, the electronic device 101 may continuously measure biometric information through continuous measurement 1520. For example, the electronic device 101 may continuously measure biometric information during a user's sleep. The electronic device 101 may identify whether the user is sleeping. The electronic device 101 may enter continuous measurement 1520 mode based on identifying that the user is sleeping. The electronic device 101 in continuous measurement 1520 mode may continuously measure biometric information. The electronic device 101 may identify that the user's sleep has ended. The electronic device 101 may terminate continuous measurement 1520 mode based on identifying that the user's sleep has ended. The electronic device 101 may provide feedback regarding contact after continuous measurement 1520 ends. The electronic device 101 may provide biometric information obtained by continuous measurement 1520 after continuous measurement 1520 ends.

In an embodiment, in case of performing continuous measurement 1520, the electronic device 101 may repeatedly perform the operation of obtaining biometric information according to the contact state after determining whether the electronic device 101 and at least a portion of a user's body are in contact. Continuous measurement 1520 may include nth measurement (n is a natural number of 2 or more). The electronic device 101 may obtain a plurality of biometric information by continuous measurement 1520. The electronic device 101 may obtain continuous biometric information based on the plurality of biometric information obtained by continuous measurement 1520. For example, the electronic device 101 may obtain an average value of AGE values obtained by continuous measurement 1520. The electronic device 101 may provide the obtained biometric information after continuous measurement 1520 ends (e.g., see FIG. 16). The electronic device 101 may provide feedback regarding contact after continuous measurement 1520 ends. The feedback may include a ratio of the number of times the electronic device is determined to contact at least a portion of the user's body to the number of times the electronic device 101 determines whether it contacts at least a portion of the user's body. The feedback may include a feedback message 1530. For example, the feedback message 1530 may include a message such as “The measurement success rate for this sleep is 25%” and/or “Please tighten the strap for better contact with the skin during the next sleep.”

For example, continuous measurement 1520 may include a first measurement, a second measurement, and a third measurement. During the first measurement, the electronic device 101 may determine whether the electronic device 101 and at least a portion of a user's body are in contact using the first sensor. The electronic device 101 may identify a contact state in which the electronic device 101 and at least a portion of a user's body are in contact, or a non-contact state in which the electronic device 101 and at least a portion of a user's body are not in contact. The electronic device 101 may drive the second sensor to obtain first biometric information based on identifying a contact state. The electronic device 101 does not drive the second sensor for obtaining first biometric information based on identifying a non-contact state. During the second measurement, the electronic device 101 repeats the same process as in the first measurement. The electronic device 101 may determine whether the electronic device 101 and at least a portion of a user's body are in contact using the first sensor. The electronic device 101 may identify a contact state in which the electronic device 101 and at least a portion of a user's body are in contact, or a non-contact state in which the electronic device 101 and at least a portion of a user's body are not in contact. The electronic device 101 may drive the second sensor to obtain second biometric information based on identifying a contact state. The electronic device 101 does not drive the second sensor for obtaining first biometric information based on identifying a non-contact state. During the third measurement, the electronic device 101 repeats the same process as in the first measurement. For example, during the first and second measurements, the electronic device 101 may identify a contact state, and during the third measurement, the electronic device 101 may identify a non-contact state. In this case, the electronic device 101 obtains first biometric information by the first measurement and second biometric information by the second measurement but fails to obtain third biometric information by the third measurement. The electronic device 101 may obtain continuous biometric information based on the obtained first biometric information and second biometric information.

FIG. 16 is a diagram illustrating example biometric information provided by an electronic device (e.g., the electronic device 101 of FIG. 1) according to an embodiment.

In FIG. 16, the electronic device 101 may provide biometric information. The electronic device 101 may provide biometric information through a display (e.g., the display module 160 of FIG. 1). The electronic device 101 may provide biometric information through a user interface screen. For example, the biometric information may include an AGE value (AGE value or AGE Index). For example, the user interface screen may include an object 1610 related to biometric information and/or a graph 1620 related to biometric information. For example, the object 1610 may include the type of biometric information (e.g., AGE Index) and the value of the biometric information (e.g., 50). For example, the graph 1620 may be a graph illustrating the value of biometric information measured on the vertical axis with date on the horizontal axis. In an embodiment, the electronic device 101 may display the object 1610 after one-time (e.g., a single) measurement. The object 1610 may include the type and value of biometric information measured by one-time measurement. In an embodiment, the electronic device 101 may display the object 1610 and/or the graph 1620 after continuous measurement. The object 1610 may include the type and value of biometric information measured by continuous measurement.

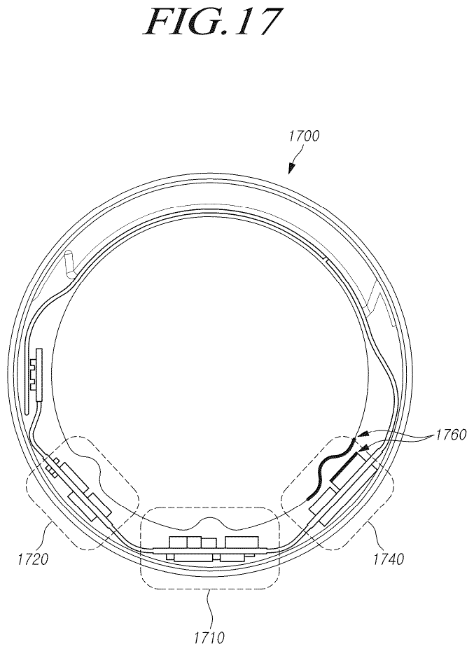

FIG. 17 is a diagram illustrating an example ring-type electronic device according to an embodiment.

In FIG. 17, the electronic device 101 may be a ring-type electronic device 1700. The ring-type electronic device 1700 may include an optical sensor for measuring a user's bio-signal. According to an embodiment, the optical sensor may include a light emitter 1710, a first light receiver 1720, and a second light receiver 1740. For example, the light emitter 1710 may include at least one of a UV light emitting element, a blue light emitting element, a violet light emitting element, a green light emitting element, a red light emitting element, or an IR light emitting element.

In an embodiment, the second light receiver 1740 may include a light-filtering member 1760. The light-filtering member 1760 may be formed on the light receiving element of the second light receiver 1740. Further, the light-filtering member 1760 may be formed on a region corresponding to the light receiving element on the inner circumferential surface of the ring-type electronic device 1700.

In an embodiment, the electronic device 101 may be a wearable device. For example, the wearable device may include a watch, a ring, earphones, and/or augmented reality (AR) glasses. However, the disclosure is not limited thereto, and the electronic device 101 may be various devices such as a smartphone, a standalone device for measuring bio-signals, a refrigerator including a biometric sensor, a cooker, a smart mirror, or a beauty device.

In an example embodiment, an electronic device 101 may include a first light emitting device configured to emit light, a first light receiving device configured to receive light of a first wavelength band, a second light receiving device configured to receive light of a second wavelength band, a memory 130 including at least one storage medium storing instructions, and at least one processor 120, comprising including processing circuitry, wherein at least one processor, individually and/or collectively, is configured to execute the instructions and to cause the electronic device to: receive first light by the first light receiving device in response to light emission by the first light emitting device, receive second light by the second light receiving device in response to light emission by the first light emitting device, and determine whether the electronic device contacts at least a portion of a body of a user based on the first light and the second light.

In an example embodiment, the determining whether the electronic device contacts at least a portion of the body of the user includes: determining that the electronic device contacts at least a portion of the body of the user based on a ratio of an intensity of the first light to an intensity of the second light being equal to or greater than a threshold.

In an example embodiment, the electronic device may comprise a wearable device, and wherein the first light includes light generated as a result of an interaction between the light emitted by the first light emitting device and the body of the user wearing the wearable device, and the second light includes light reflected from the body of the user wearing the wearable device by light emitted by the first light emitting device.

In an example embodiment, at least one processor, individually and/or collectively, is configured to cause the electronic device to provide feedback for improving contact based on determining that the electronic device does not sufficiently contact at least a portion of the body of the user.

In an example embodiment, at least one processor, individually and/or collectively, is configured to cause the electronic device to drive a biometric sensor to obtain biometric information based on determining that the electronic device contacts at least a portion of the body of the user.

In an example embodiment, at least one processor, individually and/or collectively, is configured to cause the electronic device to repeat an attempt to obtain biometric information, wherein the attempt to obtain biometric information may include driving a biometric sensor to obtain biometric information based on determining that the electronic device contacts at least a portion of the body of the user, and determining whether the electronic device contacts at least a portion of the body of the user based on determining that the electronic device does not contact at least a portion of the body of the user.

In an example embodiment, at least one processor, individually and/or collectively, is configured to cause the electronic device to provide feedback regarding contact after the repeating of the attempt to obtain biometric information is terminated.

In an example embodiment, the electronic device 101 may comprise a light filter on the first light receiving device.

In an example embodiment, a peak wavelength of light emitted by the first light emitting device may be in a range of 360 nm to 370 nm.

In an example embodiment, the first wavelength band may include a range of 480 nm to 980 nm, and the second wavelength band may include a range of 340 nm to 980 nm.

In an example embodiment, a method of controlling an electronic device 101 may comprise receiving first light by a first light receiving device in response to light emission by a first light emitting device, receiving second light by a second light receiving device in response to light emission by the first light emitting device, and determining whether the electronic device contacts at least a portion of a body of a user based on the first light and the second light.

In an example embodiment, the determining whether the electronic device contacts at least a portion of the body of the user may include determining that the electronic device contacts at least a portion of the body of the user based on a ratio of the intensity of the first light to the intensity of the second light being equal to or greater than a threshold.

In an example embodiment, the electronic device may comprise a wearable device, the first light includes light generated as a result of an interaction between the light emitted by the first light emitting device and the body of the user wearing the wearable device, and the second light includes light reflected from the body of the user wearing the wearable device by light emitted by the first light emitting device.

In an example embodiment, the method may further comprise providing feedback for improving contact based on determining that the electronic device does not sufficiently contact at least a portion of the body of the user.

In an example embodiment, the method may further comprise driving a biometric sensor to obtain biometric information based on determining that the electronic device contacts at least a portion of the body of the user.

In an example embodiment, the method may further comprise repeating an attempt to obtain biometric information. The attempt to obtain biometric information may include driving a biometric sensor to obtain biometric information based on determining that the electronic device contacts at least a portion of the body of the user, and determining whether the electronic device contacts at least a portion of the body of the user based on determining that the electronic device does not contact at least a portion of the body of the user.

In an example embodiment, the method may further comprise providing feedback regarding contact after the repeating of the attempt to obtain biometric information is terminated.

In an example embodiment, the electronic device may include a light filter on the first light receiving device.

In an example embodiment, a peak wavelength of light emitted by the first light emitting device may be in a range of 360 nm to 370 nm.

In an example embodiment, the first wavelength band may include a range of 480 nm to 980 nm, and the second wavelength band may include a range of 340 nm to 980 nm.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), the element may be coupled with the other element directly (e.g., wiredly), wirelessly, or via a third element.

As used herein, the term “module” may include a unit implemented in hardware, software or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

According to various embodiments, each component (e.g., a module or a program) of the above-described components may include a single entity or multiple entities. Some of the plurality of entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been illustrated and described with reference to various example embodiments, it will be understood that the various example embodiments are intended to be illustrative, not limiting. It will be further understood by those skilled in the art that various modifications, alternatives and/or variations of the various example embodiments may be made without departing from the true technical spirit and full technical scope of the disclosure, including the appended claims and their equivalents. It will also be understood that any of the embodiment(s) described herein may be used in conjunction with any other embodiment(s) described herein.

What is claimed is:

1. An electronic device comprising:
a first light emitting device configured to emit light;
a first light receiving device configured to receive light of a first wavelength band;
a second light receiving device configured to receive light of a second wavelength band;
a memory including at least one storage medium storing instructions; and
at least one processor, comprising processing circuitry, wherein at least one processor, individually and/or collectively, is configured to execute the instructions and to cause the electronic device to:
receive first light by the first light receiving device in response to light emission by the first light emitting device;
receive second light by the second light receiving device in response to light emission by the first light emitting device; and
determine whether the electronic device contacts at least a portion of a body of a user based on the first light and the second light.

2. The electronic device of claim 1, wherein the determining whether the electronic device contacts at least a portion of the body of the user includes: determining that the electronic device contacts at least a portion of the body of the user based on a ratio of an intensity of the first light to an intensity of the second light being equal to or greater than a threshold.

3. The electronic device of claim 1, wherein the electronic device comprises a wearable device, and wherein the first light includes light generated as a result of an interaction between the light emitted by the first light emitting device and the body of the user wearing the wearable device, and the second light includes light reflected from the body of the user wearing the wearable device by light emitted by the first light emitting device.

4. The electronic device of claim 1, wherein at least one processor, individually and/or collectively, is configured to cause the electronic device to provide feedback for improving contact based on determining that the electronic device does not sufficiently contact at least a portion of the body of the user.

5. The electronic device of claim 1, wherein at least one processor, individually and/or collectively, is configured to cause the electronic device to drive a biometric sensor to obtain biometric information based on determining that the electronic device contacts at least a portion of the body of the user.

6. The electronic device of claim 1, wherein at least one processor, individually and/or collectively, is configured to cause the electronic device to:
repeat an attempt to obtain biometric information, wherein the attempt to obtain biometric information includes:
driving a biometric sensor to obtain biometric information based on determining that the electronic device contacts at least a portion of the body of the user; and
determining whether the electronic device contacts at least a portion of the body of the user based on determining that the electronic device does not contact at least a portion of the body of the user.

7. The electronic device of claim 6, wherein at least one processor, individually and/or collectively, is configured to cause the electronic device to provide feedback regarding contact after the repeating of the attempt to obtain the biometric information is terminated.

8. The electronic device of claim 1, comprising a light filter on the first light receiving device.

9. The electronic device of claim 1, wherein a peak wavelength of the light emitted by the first light emitting device is in a range of 360 nm to 370 nm.

10. The electronic device of claim 1, wherein the first wavelength band includes a range of 480 nm to 980 nm, and the second wavelength band includes a range of 340 nm to 980 nm.

11. A method of controlling an electronic device, comprising:
receiving first light by a first light receiving device in response to light emission by a first light emitting device;
receiving second light by a second light receiving device in response to light emission by the first light emitting device; and
determining whether the electronic device contacts at least a portion of a body of a user based on the first light and the second light.

12. The method of claim 11, wherein the determining whether the electronic device contacts at least a portion of the body of the user includes determining that the electronic device contacts at least a portion of the body of the user based on a ratio of an intensity of the first light to an intensity of the second light being equal to or greater than a threshold.

13. The method of claim 11, wherein the electronic device comprises a wearable device, and wherein the first light includes light generated as a result of an interaction between the light emitted by the first light emitting device and the body of the user wearing the wearable device, and the second light includes light reflected from the body of the user wearing the wearable device by light emitted by the first light emitting device.

14. The method of claim 11, further comprising providing feedback for improving contact based on determining that the electronic device does not sufficiently contact at least a portion of the body of the user.

15. The method of claim 11, further comprising driving a biometric sensor to obtain biometric information based on determining that the electronic device contacts at least a portion of the body of the user.

16. The method of claim 11, further comprising repeating an attempt to obtain biometric information, wherein the attempt to obtain biometric information includes:
driving a biometric sensor to obtain biometric information based on determining that the electronic device contacts at least a portion of the body of the user; and
determining whether the electronic device contacts at least a portion of the body of the user based on determining that the electronic device does not contact at least a portion of the body of the user.

17. The method of claim 16, further comprising providing feedback regarding contact after the repeating of the attempt to obtain the biometric information is terminated.

18. The method of claim 11, wherein the electronic device includes a light filter on the first light receiving device.

19. The method of claim 11, wherein a peak wavelength of the light emitted by the first light emitting device is in a range of 360 nm to 370 nm.

20. The method of claim 11, wherein the first wavelength band includes a range of 480 nm to 980 nm, and the second wavelength band includes a range of 340 nm to 980 nm.
